SEQUENCE LISTING (20 sequences)

SEQ ID NO: 1 - protein, 1273 aa, Coronaviridae
  SARS-CoV-2 S Protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - DNA, 3500 bp, Artificial Sequence
  pVAX10
tgggcttttgctggccttttgctcacatgttcttgactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagacccccatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatc

SEQ ID NO: 3 - protein, 15 aa, Coronaviridae
  Signal peptide S protein
MFVFLVLLPLVSSQC

SEQ ID NO: 4 - protein, 28 aa, Homo sapiens
  28 aa of C3d
KFLTTAKDKNRWEDPGKQLYNVEATSYA

SEQ ID NO: 5 - protein, 112 aa, Artificial Sequence
  3C3d
KFLTTAKDKNRWEDPGKQLYNVEATSYAGSGGGGSGGGGSGSKFLTTAKDKNRWEDPGKQLYNVEATSYAGSGGGGSGGGGSGSKFLTTAKDKNRWEDPGKQLYNVEATSYA

SEQ ID NO: 6 - protein, 22 aa, Artificial Sequence
  2A peptide P2a
GSGATNFSLLKQAGDVEENPGP

SEQ ID NO: 7 - protein, 21 aa, Artificial Sequence
  2A peptide T2a
GSGEGRGSLLTCGDVEENPGP

SEQ ID NO: 8 - protein, 419 aa, Coronaviridae
  N protein YP_009724397.2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 9 - protein, 75 aa, Homo sapiens
  ubiquitin
MQIFVKTLTGKTITLEVEPSDTIENVKAKIQDKEGIPPDQQRLIFAGKQLEDGRTLSDYNIQKESTLHLVLRLRG

SEQ ID NO: 10 - protein, 27 aa, Artificial Sequence
  T4 Fibritin foldon trimerization domain
GYIPEAPRDGQAYVRKDGEWVLLSTFL

SEQ ID NO: 11 - DNA, 5154 bp, Artificial Sequence
  SCV-3
gctagcgccgccaccatgttcgtcttcttagtgcttctgccccttgtttcatctcagtgcgtaaatctcaccaccagaacccagctgcctcccgcctacacgaactcctttactaggggggtatattaccctgacaaggtattcagatcctcagtactgcattccacacaggacctcttcctccctttcttcagcaatgtaacctggtttcacgccattcatgtctccgggaccaacggcacgaagaggtttgacaaccccgtcctaccctttaatgacggtgtgtactttgcgtcgacagaaaagagtaatatcattcgcggctggatttttggtacaaccttagattctaagacgcagtcactccttatagtgaacaatgccacaaatgttgttatcaaggtgtgtgaatttcaattctgcaatgatcctttcctgggtgtgtactaccataagaataacaaaagttggatggaatcagaatttagagtttactcttcagctaacaattgcactttcgagtatgtttcccagcctttcctcatggatctggaggggaaacaagggaatttcaaaaatctgcgcgaattcgtgttcaaaaatatagatggatatttcaagatctacagcaaacacacacctatcaaccttgttcgagatctgccccagggattttctgccctggaaccactcgtggacctgcccatcggtattaacatcactcgattccagacactactggccctccaccggagctacctgacccctggggattcttctagtgggtggacagcaggcgcagctgcttactacgtgggctatttgcagcctcggaccttcttactgaaatacaatgaaaatggcaccatcacagatgctgtggactgtgccttagaccctctctccgaaaccaaatgtacccttaagagcttcacagtcgaaaaagggatttaccaaacgtcgaacttcagggtgcagcccactgagagcatcgtcagatttcccaatattacgaacctgtgtcccttcggagaagtgttcaacgctactcgatttgcctctgtgtacgcatggaatcgcaaaaggatctcgaattgcgtagccgactactccgttctgtataattccgcatcctttagcacttttaaatgctacggggtcagccctactaaactgaacgacctctgcttcaccaacgtctacgctgattcttttgtcatccgtggggatgaagtgaggcagatagccccaggtcagactggaaagatcgcggattataattacaaactgccagacgacttcactggctgcgtgatcgcctggaacagtaacaacctcgacagcaaagtgggcggaaactacaactatttatatcgcttgtttagaaagtccaacttgaagccttttgaaagagacatttctactgagatttaccaggctgggtcaacaccctgtaacggcgtggaaggctttaactgttacttccccttacaaagctatggcttccagccaaccaacggagttgggtatcagccctacagggtcgtcgtgttgagtttcgagctcttacatgcacctgccaccgtctgcggccccaagaagtctacgaacctggtgaagaacaagtgtgtcaatttcaactttaatggactaactgggacaggcgtgctgacggagtcaaacaagaaattcctgccatttcagcagtttggtcgggacattgcagacacaactgacgcggtgcgggaccctcagacgcttgaaatattagacataacaccttgtagctttggcggagtgtctgtcatcaccccgggaactaacaccagcaaccaggtggcagtcctatatcaggacgtcaattgcacggaggttcccgtggcaatacacgcggaccaactcacaccgacttggagagtgtattccacaggtagcaacgtctttcaaactcgggctggttgcctgattggcgcagagcacgtgaacaacagctatgagtgtgacattcccataggcgcagggatttgtgcttcataccagactcagactaacgcccgtagtgtagccagccagtccatcattgcatacactatgtcactgggagcagagaactctgttgcctacagtaacaatagtattgctattccgacgaactttaccatcagtgtgactaccgagatcctgccagtctcgatgaccaagacctccgtagattgtaccatgtatatctgcggggacagcaccgagtgctctaatttgttgctgcagtatgggtccttctgtacgcagttgaatcgggccttgacaggcatcgctgtcgagcaggataaaaacactcaagaggtctttgcacaggttaagcagatttataagacacctcccataaaagacttcggtggctttaacttctcacaaatcttaccagacccaagtaagccaagtaaacgttctttcatcgaggacctcctctttaataaggtgacccttgctgatgcaggtttcatcaagcagtacggtgactgcctgggagacatagcagcccgggatttgatctgcgcacagaagttcaacggactaacagtgctcccacctttgctgacggatgagatgattgcccagtacacatcagctctcctggcgggcaccattacctcaggatggacctttggagcaggagctgcgctccagatcccgtttgccatgcagatggcctataggttcaacggcataggcgttacacagaacgtgctgtatgagaatcagaagctcatcgccaaccagttcaattctgccataggaaaaatccaggattccctttcttcaactgcctcagccctcgggaagctacaagatgtggtcaatcagaacgctcaggccctaaatactcttgtgaagcagctgtcgtcaaactttggggccatcagcagtgttctgaacgatatcctgtcacggctggatccaccagaggccgaggtccagattgaccgactaattaccggcaggctgcaatctcttcagacttacgtcacacagcagctgatccgagctgcagaaatcagagccagtgcaaacctggctgcaacgaaaatgtcagagtgtgtgcttggacagtcaaaaagagtagacttttgtggcaagggataccatttgatgtcctttccacaatccgctccgcatggagttgtgtttttgcatgttacctatgtccctgcccaggaaaagaactttaccacagctcccgccatatgccacgatgggaaggcccactttccacgcgagggagtcttcgtgtctaatggtactcactggtttgtgacccaacgcaatttttacgaacctcaaatcattaccacagacaatacattcgtatcaggtaactgcgatgtcgtgattggaatcgtgaataataccgtttatgacccccttcaaccagaactggactcatttaaggaagagctcgacaaatatttcaaaaaccacactagcccggatgtggatttgggcgatataagcggcatcaatgctagtgtggtgaacatccagaaggagattgaccggctaaatgaagtggccaagaacctgaatgagagcttgattgacctccaggagcttggtaaatacgaacaatatatcaagtggccatggtatatctggctgggattcattgccggcctgatcgccatcgtgatggtaacaatcatgctgtgctgcatgactagctgctgctcctgtctgaagggctgttgctcttgtggtagttgttgtaagtttgatgaagacgatagcgagccagttctcaaaggagtgaagttgcactacacaggatccggtgaaggccgtgggtcccttttaacgtgtggtgacgtagaagaaaacccaggcccgctcatgagcgacaatggaccacagaatcagcgaaacgcccccaggattacatttggcggtcccagtgactcgaccggcagtaaccagaatggggagaggtctggggcgaggtctaagcagaggaggccacaaggcctgccaaataatactgcgagctggttcacagcactgacacagcatggcaaagaggatctcaaattcccaagaggtcaaggcgttccaatcaatactaacagctctcctgatgatcagatcggctactatagacgggccacgcgacgaattcgcggaggggatggtaagatgaaggatttgtcaccacgctggtacttttattaccttgggacaggacctgaggcagggcttccatacggcgctaataaagacggcattatctgggttgccacagagggggcactgaatacccctaaggatcacatcggcactagaaaccctgcgaataacgcagccatagtgctccagttaccacagggaaccacactgcctaagggtttctatgctgaggggagcaggggaggaagtcaggcatcgtccagatccagttctagatcgcggaacagcagtcggaattccactcctggctcatcaagaggaacctcaccagccaggatggcagggaacggaggagacgcagcgctggctctgctcctcctggatagacttaaccaactggagagcaagatgtccggaaagggccagcaacaacaaggtcagaccgtgacgaagaaatcagccgccgaagctagtaaaaagccccggcagaaacggactgctacaaaggcatacaatgtcacacaagccttcggtcgcagggggccagaacaaacccaggggaatttcggtgatcaggagctgatacggcagggaacagactataagcactggccgcagatcgctcagttcgcaccctccgcaagcgcctttttcggcatgtctcgcataggaatggaggtgacgcccagtggcacatggctaacttatacaggcgccattaaacttgatgacaaagacccgaacttcaaggaccaagtcatcttgctgaacaagcatattgatgcttataagactttccctcctactgagccgaaaaaggataaaaaaaagaaagctgacgagacacaggcgcttccccagcgtcaaaagaaacagcagacagtgacgctgttgcccgctgctgatttggacgatttcagcaagcaactacagcagtccatgtccagcgctgattccacccaagcttaactcgag

SEQ ID NO: 12 - DNA, 2619 bp, Artificial Sequence
  SCV-30
gctagcgccgccaccatgttcgtgtttctggtgttactcccattggtgtctagagtgcagccgaccgaatccatcgtgcgcttccctaatatcactaatctgtgcccctttggggaagtttttaatgccactaggttcgcatctgtgtatgcatggaaccgcaagcgaattagcaactgcgtcgctgactattcagtactgtataactctgccagtttctccactttcaagtgctatggggtatcccccaccaagctcaatgacctttgctttacaaacgtgtacgccgattcctttgtgattcgaggagatgaagtgaggcagatagccccagggcagactggaaagatcgccgactacaattacaaactgccggatgatttcaccgggtgtgtgattgcttggaatagcaataatctggattctaaagtgggaggcaactataattacctatacagactgttccggaagtctaacctaaagccatttgaaagagacatttcaaccgaaatataccaagcgggctctaccccgtgtaatggcgttgagggcttcaattgctatttccctcttcagtcctacgggttccagcctacaaatggtgtcggctaccagccctacagggttgtcgtcttaagtttcgagctgctgcatgcacccgccaccgtctgtggcccaaaaaagagtactaacttggtaaaaaacaagtgtgtgaactttggatccaaatttcttaacaccgctaaggatcgtaataggtgggaggaaccagatcagcagctctacaacgttgaagccacctcatacgcaggatcgggtgggggaggaagcggcggcggaggcagcggctctaagtttctgaacacagcaaaggacaggaatcgttgggaggaacctgaccagcagctctacaacgtcgaggcaaccagttacgcgggtagtggagggggcgggtctggaggcggaggaagcggttccaagtttctcaacacagctaaagaccgcaaccggtgggaggagccggaccagcaattatataatgtggaggccacatcgtatgccgggagtggcgagggcaggggttcactgctgacttgtggagatgtggaagagaacccagggccactgatgcagatctttgtcaagacacttacaggaaaaaccattaccctggaggtggagccaagtgacacaattgagaacgttaaggcaaaaatccaggacaaggagggcatcccccctgaccaacaacgacttatatttgcaggtaagcaactagaagatggccgcacactttcagactacaacatccaaaaagagtctacactccacctggtcttgagattaagaggaatgtcagacaacgggccgcaaaaccagcggaatgctcccagaatcactttcggggggcctagtgactcaaccggtagcaaccagaacggggagcgttccggcgccaggtcaaagcagcgtcggcctcagggcctccccaacaacactgccagctggttcactgctttaacccagcatggtaaagaagacctgaagttcccccgagggcagggtgtacctattaacacgaacagctctcctgatgatcagatcggctattatcgaagagccacaaggagaatacgcggaggcgacgggaaaatgaaagacctctcccccaggtggtacttctactatctgggcacggggcctgaggccgggctgccatacggcgcgaataaagatggaatcatctgggtagctacggagggcgcattaaatactcccaaggaccatattggaaccaggaatccagctaacaatgcagctattgttttgcagctcccccaggggacaactctgcccaagggattctatgctgaaggtagccgtggaggcagtcaggcgtctagtcggtcctctagtagaagccgaaattctagccggaatagcaccccaggttcctctcggggcacatcacctgctcggatggcaggtaacgggggagatgctgccctggctctcctgctgttggatagattgaatcagttggagtctaagatgtccggcaagggacaacagcagcaaggtcagacagtcacgaagaaaagcgcagcggaggccagcaagaaacctcgccagaagagaacagccaccaaagcctacaatgtgacccaggccttcggccggcgggggcccgaacagactcaaggaaattttggtgaccaggaactgatacgacaggggacagattataagcactggcctcaaatcgctcagttcgctccttctgccagtgccttctttggtatgagcaggatcggcatggaagttactccttccggcacgtggcttacttacacaggagccatcaaacttgatgacaaagatccaaactttaaggatcaagtcatcttactaaataagcacattgatgcttataaaactttcccacccaccgaaccaaaaaaggacaaaaaaaaaaaggccgacgagacccaagctttgcctcagcgccagaagaagcagcagaccgtgacgctcttgccagcagcagacctggacgatttttcgaaacagcttcagcagtccatgtcgagcgcggactcaacacaagcataactcgag

SEQ ID NO: 13 - DNA, 5787 bp, Artificial Sequence
  SCV-42
gctagcgccgccaccatgttcgtgttcttggtgctcttaccactggtctcctcacagtgcgtgaacctgacaacacgaacccaactgccaccagcatacacgaattcttttacccgaggtgtgtattatcctgacaaagtctttagaagttcagtcctccattccactcaagatttgtttctcccgttcttctcgaatgtgacatggtttcatgctattcatgtgtcggggacaaatggcaccaagcggtttgacaatcctgtcttacctttcaatgatggggtttatttcgcatcaacagaaaagagcaacattatacggggctggatattcgggaccaccctggattccaaaacccagtctctgctaattgtgaataacgccacaaatgtagttatcaaagtgtgtgagttccagttttgcaacgatccattcttgggtgtctactatcacaagaacaataaatcatggatggagagcgagtttagggtctattcttctgcaaacaactgtacgttcgagtacgtctcccagccattcctcatggacctggagggcaaacaggggaacttcaaaaatcttagagaattcgtcttcaaaaacatcgatgggtattttaagatctacagcaaacatacccccatcaatctcgtgcgggaccttcctcagggcttttccgcacttgaacctctcgtcgaccttcccatcggaatcaatattacaaggttccagacactactcgcccttcacagatcctacctgacgccaggcgattcctcctctggctggacagcgggagccgctgcctattatgtgggctatttgcagcctcgcacattcctcttgaaatacaacgagaatgggaccattacagatgcagtggactgtgcacttgaccctctgtctgagacaaagtgtacactgaaatccttcacggtggagaagggtatctaccagacttctaacttccgggttcagcccactgagtccatagtccggttcccaaacataactaacctatgcccatttggggaggttttcaatgctacccgcttcgcctcagtttatgcttggaacaggaagagaattagcaactgtgtggccgactatagtgtgctgtacaactccgccagcttctccaccttcaaatgctatggtgtgtcacctaccaaactgaatgatctgtgctttacaaatgtttacgctgatagttttgtaatcagaggtgacgaagtaagacagattgcccccggacaaactgggaagatcgcggattacaattacaaacttcccgacgactttacgggctgcgtgatcgcctggaatagcaataatctggacagcaaggtgggcgggaattataactacctctaccgcctcttcaggaaaagtaacctgaagccctttgaacgtgatataagcactgagatataccaagctggcagcacgccttgtaacggagtggaaggattcaactgctacttccctttacaaagttatggcttccagccaaccaacggggtgggttatcagccatacagggttgttgtgctaagttttgagttattacatgcacctgctactgtctgcggcccaaaaaagtccaccaacctggttaaaaacaaatgtgtcaattttaatttcaatggcctaacaggtacgggtgtcttgactgaaagtaataaaaaatttcttccatttcagcagtttgggagagacatcgcggacacaaccgatgctgtgagagatccgcagacactggagattttggacatcaccccgtgtagctttggcggtgtatctgtgataactcctgggactaatacaagcaaccaggttgccgtgctgtatcaagacgtgaactgcactgaggtccctgtggcaatccatgctgaccagctgacgcccacctggagggtttactcgaccggaagtaatgtattccaaactcgggcaggctgcctcatcggagcagaacacgtgaacaactcctatgagtgtgatatacccatcggtgcaggaatttgtgccagctaccagacacagacaaacagcccagggagcaagttcctgaacacggccaaggatcgaaaccggtgggaggaaccagatcaacaattgtataacgttgaggccacgtcttacgccggtagtggcgggggtgggtcaggcggtggaggaagcggatcaaagttcctcaatactgcgaaggacaggaaccgctgggaagagccggaccagcagttgtacaatgtggaagccacaagctatgctggaagtggtggcggaggctcaggaggagggggatcggggtctaagtttctgaacactgcaaaggacagaaataggtgggaggagcctgaccagcaactgtacaacgtggaagcaacaagctacgccggctcaggggagggaagagggagcctcctgacatgtggggacgtagaggagaaccccgggccgctcatgcaaattttcgtaaagactctgacaggcaagacgatcacactggaagtcgagccatctgacaccatcgaaaatgtaaaagccaaaatacaagacaaagaaggaattcccccagaccaacagcgactcatctttgcaggcaagcagctggaggacggacggaccttgtctgactacaatattcagaaggagtctactttgcatctggttcttcggctaagaggcatgagtgacaacggcccacaaaaccaaaggaatgccccgaggatcaccttcggcggaccctccgatagcacaggcagtaaccagaatggggaaaggagtggagccaggtcaaaacagcgtcggccacagggactgcccaacaacaccgcttcctggttcacagcactgacccagcatgggaaagaggatttgaaattccctcgtggtcaaggtgtccccattaacacgaactcatcccctgatgaccagatcggttactacagaagagcaaccaggaggatacgcggcggagacggcaaaatgaaggatctgtctccgcggtggtatttctactaccttggcaccggccctgaggctggactgccttacggagcaaacaaagatggaattatctgggtggccaccgagggtgctctcaacacgcccaaggaccacataggtactcgcaacccagccaataatgctgccatcgtgctgcagctgcctcagggaacaacacttcccaaaggattctatgctgagggcagccgcggaggctcgcaggcctcgtccaggtccagttcaaggtctcgaaattcatcccggaactctactccaggttctagtagaggaacaagtccggcaaggatggccgggaatggtggagatgccgctctggccctcctactacttgacagattaaaccagctggaaagcaaaatgagtggaaagggccagcagcaacagggccagacagtgacaaagaagtcagctgcggaggcctccaagaagcccaggcagaaacgcactgctactaaagcttataatgtcacccaagctttcgggcgcaggggccccgaacaaacacagggtaattttggagatcaggaactcattcgtcagggcacggattataaacactggccccagattgcccagtttgcaccatctgccagcgccttttttgggatgagtcgcattggaatggaagttactccttctggcacctggctgacatatactggagccattaagctggacgacaaggatcccaatttcaaggaccaagtaatactgctcaataagcacattgatgcctacaaaacctttccgcccactgagcccaagaaagataagaagaagaaagcagatgagacccaggctctcccccagagacagaagaaacagcagacggttaccctgctgcctgccgcggacctggatgactttagcaagcaacttcagcagtccatgtcgtccgctgatagtactcaggccgggtctggggaggggcggggcagcctgcttacttgtggtgatgttgaagagaaccctggaccccttagtgtggcatcacaatctattattgcatataccatgtccctcggcgcggagaacagcgtggcctacagtaacaactccatagccattcctaccaactttaccatctctgtcaccacagaaattctccccgtgtccatgaccaagacctcagtggactgcactatgtacatttgcggcgattccaccgagtgctccaacttgcttctgcagtacggatcgttctgtacacaattgaatagagccctaaccggcatcgctgtcgaacaggataagaacacccaagaagttttcgcccaggtgaagcagatttataagacaccccctattaaggacttcggaggtttcaacttcagccagatcctgcctgatccatccaagccttccaagcggtccttcatcgaggaccttcttttcaacaaggtcaccttagcggacgcagggtttataaagcaatatggagattgccttggtgatatcgctgcccgagacctgatctgcgctcagaaatttaatggtttaacggtgttgccaccattattgacagacgagatgatcgcacagtacactagtgcgctgctggctgggacaatcacatctggatggacgtttggggctggtgcagccctccagatcccttttgccatgcaaatggcgtaccgtttcaacggcatcggcgtgactcagaacgtcctatatgagaatcagaagcttattgctaaccagttcaactccgctattggtaagattcaggacagcctcagcagcactgcgtctgcgctgggcaagttgcaagacgtggtgaatcagaatgcacaggccttaaacacactggttaaacaactgtcctcgaacttcggggctatcagctctgtcctcaatgacatcctttccagactcgacccacccgaggcagaagtccagatcgatcgactgatcacaggcagactgcagagcctgcagacctatgtcacccagcagctcatacgagctgctgagatccgagcttctgcaaacctggccgcaactaagatgtctgaatgtgtcctgggtcagagcaagcgggtggatttttgtgggaaaggctaccacctaatgtcatttccacagtctgcccctcacggcgtggtatttttgcacgtaacctacgtgccagcgcaagagaagaactttaccaccgccccagcaatctgccatgatggaaaggcccacttcccccgtgaaggggtgtttgtatccaatggaactcactggttcgtgacacagaggaacttttatgaaccacagatcatcacaaccgacaacacctttgtgtctggtaattgtgacgtggtaattgggattgtaaataacactgtgtatgatcctttacaaccagaactggattcatttaaagaagaattggacaagtactttaagaaccacactagccccgatgttgatttaggagacatctcaggcatcaacgctagtgtcgtaaatattcagaaggagatcgaccgcctgaatgaagtcgctaaaaatctcaatgagagtctgattgacttgcaggagctgggcaaatacgagcagtatatcaagtggccatggtacatctggttggggtttatagctggtctgatcgctatcgtgatggtgactataatgctgtgctgcatgaccagctgttgcagttgcctaaagggatgctgttcctgtggcagctgttgcaagtttgatgaagatgattcagaacccgtccttaagggggttaaattgcactacacctaactcgag

SEQ ID NO: 14 - DNA, 5427 bp, Artificial Sequence
  SCV-53
gctagcgccgccaccatggatgatcagcgcgatctaatttctaaccatgaacaattgcccattttaggcaacagacctagagagccagagcgttgctctcgatcgcagtgtgtcaacctgactacccgcacccaactcccaccagcttacacaaatagctttaccaggggtgtctattaccctgataaagtgtttcgtagctccgtcctccatagcacacaagatttgttcctgcccttcttctcgaatgtgacttggttccacgccatccatgtgtcaggaacaaacggaacgaaaaggtttgacaacccagtgttgcccttcaatgacggcgtttactttgcgagtaccgagaagagcaacattatccgaggctggatcttcgggacaactctcgacagcaagactcagtcgttgctcattgttaacaatgcaaccaacgtggtcatcaaggtgtgtgagttccagttctgtaatgatccgtttctgggggtttactaccacaaaaataataaatcttggatggagtcagagttccgggtgtattcttctgcaaataactgcacatttgaatatgttagtcagcctttcctcatggacttggaaggcaagcaggggaacttcaaaaatctcagagagtttgtgtttaagaatattgatggttacttcaagatctactccaaacacactcctataaaccttgtgcgggatctacctcaaggcttctcagcactggagcctttagttgacctccccattggcattaacattactaggtttcaaacacttctggccctacaccggagctaccttacccctggtgattcctcaagtggatggacagctggggcagcagcttactatgtgggatacctgcagccaagaactttcttgttgaagtacaatgagaacggaaccatcacggatgccgtggattgtgcattggatcccttgagtgaaaccaaatgtacactcaagtctttcacagttgaaaaaggaatataccaaacctccaacttccgagtgcagccaacagaatccattgttcggttccccaatatcacgaatctctgcccatttggggaggtttttaatgccacaaggtttgcctccgtgtatgcctggaacaggaagaggatctctaattgtgtagctgactactcagtcctatacaactcggcctctttttccacttttaagtgctacggcgttagtcctaccaaacttaacgacctgtgttttaccaatgtttacgcagacagctttgtgatccggggggacgaggttaggcagatagctcctggtcaaacaggtaagatcgccgattacaactataaactgcctgacgacttcacaggctgtgtgatcgcctggaattcgaacaacctggattccaaggttggcggcaattacaactacctgtaccgtctcttcagaaaaagcaatctcaagccctttgagagggatataagcactgagatctaccaggccggtagcactccctgcaacggcgtggaaggattcaactgctattttcctttgcaatcctatggttttcagccgactaatggagtgggctaccaaccctatagggtggttgtcttatcgtttgagctcctacatgctccagccacagtgtgcgggccaaagaagtccaccaaccttgtgaagaacaaatgcgtgaacttcaacttcaacgggctcactggaactggggtgctgacagagtccaacaaaaagtttctacccttccaacaattcggcagggacattgccgatacaactgacgcagttcgcgaccctcagacacttgagatcttagatataacgccctgctcctttggaggcgtctccgttattacccctggcaccaacacaagtaaccaggtggcggtgctctaccaggatgtgaactgtacagaagtacctgtggctatccacgctgaccaactgaccccaacctggagagtgtacagcacgggatccaatgtcttccagacaagagcaggatgcttgataggcgccgagcacgtcaacaacagttatgagtgtgatatccccatcggagctggcatctgcgcatcatatcagacccagacgaatgcccgttctgtagccagccagtctatcattgcctataccatgtcactgggagcagaaaactctgtggcatacagtaacaacagcatagccattcctaccaactttacaatcagtgtcaccaccgaaatcctgccggtgtccatgactaagaccagcgtggactgtactatgtacatttgcggcgactctactgagtgctcaaatctgctgctacagtatggctcattttgcacgcagctaaaccgggccttaactggcattgctgtggagcaggacaagaatactcaagaggtgttcgcccaagttaagcagatctacaaaacaccccccatcaaagactttggtggctttaacttctctcaaatacttcccgatcctagcaagccgtctaagaggtctttcatcgaagatcttttgtttaacaaagtcaccctggcagatgccggttttattaagcagtatggggactgcctgggagacatagctgcgagagacctgatctgtgctcagaagttcaatggcctgactgtactccctccattgctaaccgatgagatgatagcacagtacacttctgccctgctggctggcacaatcacctcaggctggaccttcggagccggtgccgccttgcagatcccattcgcaatgcagatggcttaccgatttaatgggatcggagttacccagaacgtcctgtatgagaaccaaaaattgattgctaatcagttcaacagcgccatcggcaagatccaggatagcttgtcaagtacagcctccgctctgggaaagctgcaggacgtcgtgaatcagaatgctcaggcacttaacacactcgtgaaacagctcagttctaatttcggagctatttccagtgtccttaatgacatcctgtcacgcctggatccccctgaagctgaagtgcagatcgaccgattaattaccgggcgtctgcagtctcttcagacttatgtcactcagcaattaatcagggcggccgaaattcgggcatccgccaacttagcggccacaaagatgtcagaatgtgtactgggccaaagtaaaagagtagatttttgtggaaaaggatatcatctgatgtctttccctcagagcgcccctcatggggtcgtattcttacacgtgacctatgtcccagcccaggagaagaacttcacgacagctcccgcgatctgtcatgacgggaaagcgcatttccctcgggagggagtatttgtttcaaacggaactcactggtttgtcacacagaggaacttttatgaacctcagataattacaacggataacacctttgtctccggaaattgtgacgtagtgataggtattgtaaataatacagtctatgacccattacagcctgagctcgacagtttcaaagaagaactggacaaatacttcaagaaccacaccagtccagatgtggatctgggagacatctccggcatcaatgcttctgtggtgaacatccagaaggaaattgaccgcttgaatgaagtcgccaaaaacctgaacgaaagcttaattgatctgcaggaactggggaaatatgagcagtatataaagtggccctggtacatctggctgggattcattgctggtctgatcgcgattgtgatggtgacgatcatgctgtgttgcatgacatcctgttgttcttgtctgaaaggctgctgctcctgtggtagctgctgcaaatttgatgaagacgatagcgagccggtcctgaagggtgttaagctgcattatactgggagcggagagggtagggggtcgttgctgacctgcggagatgttgaggagaatccaggccccatgcagatctttgtgaaaaccctgaccggtaagaccatcactctggaggtggagccctcggacaccattgaaaatgtaaaggccaagattcaggacaaggagggcattccacctgaccagcagcgacttatttttgcagggaagcagctggaggatgggcgcactctgtcggattataacatacaaaaggagagtacactccacctggtgctccggctgcgagccatgagcgacaatggaccccaaaaccagagaaacgcaccccggatcacattcggagggccgtcagattcaaccggctccaatcaaaacggagagcggtccggcgcgagaagcaagcaaagaaggccccagggtctgccaaacaacactgcatcctggttcaccgcacttactcagcacggcaaagaagaccttaagttccctaggggccagggggtacctatcaacacaaactctagccccgacgaccagataggctattatcgccgcgccactcggcggattagaggaggtgatgggaaaatgaaggacctgtcgccaagatggtatttctactacctcggaacagggccagaggcaggcctcccctacggggccaataaggatggaattatatgggtggctacagagggtgctctcaatactcccaaagaccacatagggactcggaatcccgccaacaatgcagccatcgtcctgcaactacctcaagggactaccctgccaaaaggcttctatgctgagggcagccgaggaggctctcaagccagttcccgttcaagcagccgcagcagaaattcaagcagaaattctacaccgggttcttcacgtggtaccagcccggctaggatggcgggcaacggtggggacgctgcattggctcttttacttttggacagattgaaccagctggaatccaaaatgtctggaaaagggcagcaacagcagggacagactgtgaccaagaaaagtgccgcagaggccagcaagaaacctcgccagaagagaaccgctacaaaagcctacaacgtcacccaggcatttggccggcgcgggccagaacagacacaagggaattttggggatcaggagctgataagacagggcacagactacaagcactggccacagatcgctcagtttgctccaagtgctagtgccttctttggtatgtcccggatagggatggaagtgactccttctggaacatggctcacctacaccggcgcgattaaactggatgacaaggatccaaatttcaaggaccaggtcattctcctaaataagcatatcgatgcttataagacgttcccacccacagagcccaaaaaagataagaagaaaaaggcagacgaaacccaagcactgccgcagaggcagaagaagcagcagacagtgacgctgctgccagctgcagacctggatgacttctccaaacagctccagcagtctatgagttcagcggattctactcaggcttaactcgag

SEQ ID NO: 15 - protein, 140 aa, Mus musculus
  EDL09808 (invariant chain)
MDDQRDLISNHEQLPILGNRPREPERCSRGALYTGVSVLVALLLAGQATTAYFLYQQQGRLDKLTITSQNLQLESLRMKLPKSAKPVSQMRMATPLLMRPMSMDNMLLGPVKNVTKYGNMTQDHVMHLLTVSLDPWSTRS

SEQ ID NO: 16 - DNA, 72 bp, Artificial Sequence
  SV40 DNA nuclear targeting sequence
ggtgtggaaagtccccaggctccccagcaggcagaagtatgcaaagcatgcatctcaattagtcagcaacca

SEQ ID NO: 17 - protein, 28 aa, Mus musculus
  C3d (derived from P01027.3)
KFLNTAKDRNRWEEPDQQLYNVEATSYA

SEQ ID NO: 18 - protein, 112 aa, Mus musculus
  3C3d (P01027.3)
KFLNTAKDRNRWEEPDQQLYNVEATSYAGSGGGGSGGGGSGSKFLNTAKDRNRWEEPDQQLYNVEATSYAGSGGGGSGGGGSGSKFLNTAKDRNRWEEPDQQLYNVEATSYA

SEQ ID NO: 19 - DNA, 9284 bp, Artificial Sequence
  pVAX10-SCV-53
tgggcttttgctggccttttgctcacatgttcttgactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgccgccaccatggatgaccagcgcgatctgattagcaaccatgaacagctgccaattctgggcaaccgcccacgcgaacccgaacgctgcagccgcagccagtgcgtgaacctgaccacccgcacccagctgccacccgcttataccaacagctttacccgcggcgtgtactatcctgataaagtgtttcgcagctcagtcctgcatagcacccaggatctgtttctgcctttttttagcaacgtgacctggtttcatgcaattcatgtgagcggcaccaacggcactaaacgctttgataacccagtgctgccattcaacgatggcgtgtattttgccagcaccgaaaaatcaaacattattcgcggctggatttttggcaccaccctggatagcaaaacccagagcctgctgattgtgaacaacgcaaccaacgtggtgatcaaagtgtgcgaatttcagttttgcaacgatccctttctgggcgtgtactatcacaaaaacaacaaaagctggatggaaagcgaatttcgcgtgtatagcagcgcaaacaactgcacctttgagtatgtgtcccagccatttctgatggacctggaaggcaagcaaggaaactttaaaaacctgcgcgaatttgtgtttaaaaacattgatggctattttaaaatttattccaaacatacccccattaacctggtgcgcgatctgccccaaggctttagcgccctggaacctctggtggatctgcctattggtattaacattacccgctttcagaccctgctggctctgcatcgcagctatctgacccctggcgatagcagcagcggctggaccgccggcgcagcagcttattatgtgggctatctgcaaccccgcacctttctgctgaaatacaacgaaaacggcaccattaccgatgccgtggattgcgcactggatcccctgagcgaaaccaaatgcaccctgaaaagctttaccgtggagaaaggcatttatcagacctccaactttcgcgtgcagcccaccgaaagcattgtgcgctttcctaacattaccaacctgtgcccttttggcgaagtgtttaacgcaacccgctttgccagcgtgtatgcttggaaccgcaaacgcattagcaactgcgtggctgattacagcgtgctgtataacagcgccagctttagcacctttaaatgctatggcgtgagcccaaccaagctgaacgatctgtgcttcaccaacgtgtatgcagatagttttgtgattcgcggcgatgaagtgcgccagattgctcctggccagaccggtaaaattgctgattataactataaactgccagatgattttaccggctgcgtgattgcatggaacagcaacaacctggatagcaaagtgggcggtaactataactatctgtatcgcctgtttcgcaaaagcaacctgaaaccttttgaacgcgatattagcaccgaaatttatcaggcaggtagcaccccatgcaacggcgtggaaggctttaactgctattttcctctgcagagctatggctttcaaccaaccaacggggtgggctatcaaccatatcgcgtggtggtgctgtcttttgaactgctgcatgctccagctaccgtgtgcggccccaaaaaaagcaccaacctggtgaaaaacaaatgcgtgaactttaactttaacggcctgacaggcaccggcgtgctgaccgaatcaaacaaaaaattcctgccctttcagcagtttggccgcgatattgctgatacaaccgatgcagtgcgcgatcctcagaccctggaaattctggatattaccccctgcagctttggcggcgtgagcgtgataacccctggcaccaacaccagcaaccaggtggctgtgctgtatcagggcgtgaactgcaccgaagtgcccgtggccattcatgctgatcaactgacccccacctggcgagtgtatagcacaggcagcaacgtgtttcagacccgcgccggctgcctgattggcgccgaacatgtgaacaacagctatgagtgcgatattcctattggcgctggcatttgcgcaagctatcagacccagaccaacgcccgcagcgtggccagccagagcattattgcctataccatgagcctgggcgcagaaaacagcgtggcttatagcaacaacagcattgctataccaaccaacttcaccattagcgtgaccaccgaaattctgcccgtgagcatgaccaaaaccagcgtggactgcactatgtatatttgcggcgatagcaccgaatgcagcaacctgctgctgcagtatggcagcttttgcacccagctgaaccgcgccctgactggcattgcagtggaacaggataaaaacacccaagaagtgtttgcccaggtgaaacagatttacaaaaccccccctattaaagattttggcggcttcaactttagccagatactgccagatcctagcaaacctagcaaacgcagctttatcgaagatctgctgtttaacaaagtgaccctggctgatgctggctttattaaacagtatggtgattgcctgggcgatattgccgcccgcgatctgatttgcgctcagaaatttaacggcctgaccgtgctgccccctctgctgaccgatgaaatgattgcacaatacaccagtgctctgctggctggaaccattacctcaggctggacttttggcgcaggtgccgccctgcagattccatttgccatgcagatggcctatcgctttaacggcattggcgtgacccagaacgtgctgtatgaaaaccagaaactgattgccaaccagtttaacagcgctattggcaaaatccaggatagcctgagcagcaccgccagcgcactgggcaaactgcaggatgtggtgaaccagaacgctcaggcactgaacaccctggtgaaacagctgagcagcaactttggcgccattagcagcgtgctgaacgatattctgagccgcctggaccctccagaagctgaagtgcagattgatcgcctgattacaggccgcctgcagagcctgcagacttatgtgacccagcagctgattcgcgccgctgaaatccgcgcaagcgcaaacctggctgccaccaaaatgagcgaatgcgtgctgggccagagcaaaagggtggatttttgtggcaaaggctatcacctgatgagctttccccagagcgctccacatggcgtggtgtttctgcacgtgacctatgtgcccgctcaggaaaaaaactttaccacagccccagcaatttgccatgatggcaaagctcattttccccgcgaaggcgtgtttgtgagcaacggcacccattggtttgtgacccagcgcaacttttatgaacctcagattattaccaccgataacacctttgtgagcggcaactgcgatgtggtgattggcattgtgaacaacaccgtgtatgatcccctgcagcccgaactggatagctttaaagaagagctggataaatatttcaaaaaccataccagccccgatgtggacctgggcgatattagcggcattaacgccagcgtggtgaacattcagaaagaaattgatcgcctgaacgaagtggctaaaaacctgaacgaaagcctgattgatctgcaagaactggggaaatatgaacagtatattaaatggccctggtatatttggctgggctttattgctggcctgattgccattgtgatggtgaccattatgctgtgctgcatgaccagctgctgtagctgcctgaaagggtgctgcagctgcggcagctgctgcaaatttgacgaagatgatagcgaacctgtgctgaaaggcgtcaaactgcattataccggcagcggcgaaggaagggggagcctgctgacctgtggcgatgtggaagagaaccctggcccactgatgcagatttttgtgaaaaccctgacaggcaagaccatcaccctggaagtggaacctagcgataccattgaaaacgtgaaggcaaaaatccaggataaagaagggattcctccagaccaacagcgcctgatttttgccggcaaacaactggaagatggccgcacgctgagcgattataacattcagaaggaaagcaccctgcatctggtgctgcgcctgcgcggcgtgatgagcgataacggccctcagaaccagcgcaacgcaccacgcattacctttggcggtccatcagatagcactggcagcaaccagaacggcgaacgcagcggagcccgcagcaaacaacgccgcccacagggcctgcctaacaacaccgcaagctggtttaccgccctgacccagcacggcaaagaagatctgaaatttccccgcggccagggcgtgccaattaacaccaacagcagcccagatgatcagattggctattatcgcagggccacccgccgcattcgcggaggcgatggcaaaatgaaagatctgagcccacgctggtacttttattatctgggaaccggccccgaagccggtcttccatatggcgccaacaaagatggcatcatttgggtggcaaccgaaggagctctgaacaccccaaaagatcatattggcacccgcaaccccgcaaacaacgctgcaattgtgctgcagcttcctcagggcaccaccctgccaaaaggcttttatgccgaaggcagccgcggcggcagccaggcaagctctaggagcagcagccgcagccgcaacagcagccggaacagcacccctggaagcagcaggggcaccagcccagccaggatggcaggtaacggcggcgatgctgccctggctctgctgctgctggatcgcctgaaccagctggaaagcaaaatgagcggcaaaggccagcagcagcagggccagaccgtgaccaagaaaagcgcagccgaagccagcaaaaaaccacgccagaaaagaaccgccaccaaagcctataacgtgacgcaggcttttggccgccgtggccccgaacaaacccagggcaactttggcgatcaggaactgattcgccagggcaccgattataaacattggccacagattgcccagtttgctcctagcgccagcgctttttttggtatgagccgcattggcatggaagtgaccccctctggcacctggctgacctataccggcgctattaaactggatgataaagatccaaactttaaagaccaggtgattctgctgaacaaacatattgatgcctataaaacctttcccccaaccgaacccaaaaaagataaaaagaaaaaagccgatgaaacccaggccctgccacagcgccagaaaaaacagcaaaccgtgaccctgctgcctgcagccgatctggatgatttcagcaaacagctgcaacagagcatgagcagcgcagatagcacgcaggcttaactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagttaacaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagacccccatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttgttagctcttgatccggcaaacaaaccaccgctggtagcggtggtttttttgtttgcaagcagcagattacgcgcagaaaaaaaggatctcaagaagatcctttgatc

SEQ ID NO: 20 - DNA, 281 bp, Artificial Sequence
  SV40 ori enhancer sequence
tgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagcta

(19) United States
(12) Patent Application Publication
(10) Pub. No.: US 2023/0137174 A1
(43) Pub. Date: May 4, 2023

(54) NOVEL SALMONELLA-BASED CORONAVIRUS VACCINE

(71) Applicant: NEC ONCOIMMUNITY AS, Oslo (NO)

(72) Inventors: Heinz LUBENAU, Neustadt an der Weinstrasse (DE); Marc MANSOUR, Halifax (CA)

(21) Appl. No.: 17/907,619

(22) PCT Filed: Mar. 31, 2021
(86) PCT No.: PCT/EP2021/058513

§ 371 Date: Sep. 28, 2022

(30) Foreign Application Priority Data
Mar. 31, 2020 (EP) 20167405.8

(51) Int. Cl. A61K 39/215; C12N 15/74

(52) U.S. Cl. CPC A61K 39/215; C12N 15/74; A61K 2039/523

(57) ABSTRACT

The present invention relates to a DNA vaccine comprising a Salmonella typhi Ty21a strain comprising a DNA molecule comprising a eukaryotic expression cassette encoding at least a COVID-19 coronavirus (SARS-CoV-2) spike (S) protein or a portion thereof. In particular, the present invention relates to the DNA vaccine for use in the prevention and/or the treatment of coronavirus disease 2019 (COVID-19) or a SARS-CoV-2 infection.

### FIELD OF THE INVENTION

The present invention relates to a DNA vaccine comprising a Salmonella typhi Ty21a strain comprising a DNA molecule comprising a eukaryotic expression cassette encoding at least a COVID-19 coronavirus (SARS-CoV-2) spike (S) protein or a portion thereof. In particular, the present invention relates to said DNA vaccine for use in the prevention and/or the treatment of coronavirus disease 2019 (COVID-19) or a SARS-CoV-2 infection.

### BACKGROUND OF THE INVENTION

At the end of December 2019, Chinese public health authorities reported several cases of acute respiratory syndrome in Wuhan City, Hubei province, China. Chinese scientists soon identified a novel coronavirus as the main causative agent. The disease is now referred to as coronavirus disease 2019 (COVID-19), and the causative virus is called severe acute respiratory syndrome coronavirus 2 (SARS-CoV-2). It is a new strain of coronavirus that has not been previously identified in humans.

The initial outbreak in Wuhan spread rapidly, affecting other parts of China. Cases were soon detected in several other countries. Outbreaks and clusters of the disease have since been observed in Asia, Europe, Australia, Africa and America.

The WHO in its first emergency meeting estimated the fatality rate of COVID-19 to be around 4%. Although the fatality rate seems to vary between countries and may not be accurate due to an unknown number of unreported cases the spread of SARS-CoV-2 (originally referred to as 2019 novel Coronavirus (2019-nCoV)) has become a worldwide thread and treatment of and/or vaccination against COVID-19 is desperately needed to stop further spreading of the virus.

Coronaviruses are positive-sense single-stranded RNA viruses belonging to the family Coronaviridae. These viruses mostly infect animals, including birds and mammals. In humans, coronaviruses typically cause mild respiratory infections. Since 2003 two highly pathogenic human Coronaviruses, Severe Acute Respiratory Syndrome Coronavirus (SARS-CoV) and Middle East Respiratory Syndrome Coronavirus (MERS-CoV), have led to global epidemics with high morbidity and mortality. Both endemics were caused by zoonotic coronaviruses that belong to the genus Betacoronavirus within Coronaviridae.

Like SARS-CoV and MERS-CoV, the new SARS-CoV-2 belongs to the Betacoronavirus genus. As reported by Zhou et al. (Cell Discovery (2020) 6:14) SARS-CoV-2 shares the highest nucleotide sequence identity with SARS-CoV (79.7%). Specifically, the envelope and nucleocapsid proteins of SARS-CoV-2 are two evolutionarily conserved regions, with sequence identities of 96% and 89.6%, respectively, compared to SARS-CoV. The spike protein was reported to exhibit the lowest sequence conservation (sequence identity of 77%) between SARS-CoV-2 and SARS-CoV, while the spike protein of SARS-CoV-2 only has 31.9% sequence identity with the spike protein of MERS-CoV.

Various reports relating to SARS-CoV suggest a protective role of both humoral and cell-mediated immune responses. The S protein is the most exposed protein and antibody responses against the SARS-CoV S protein have been shown to protect from SARS-CoV infection in a mouse model. While being effective antibody responses may be short-lived. In contrast, T cell responses have been shown to provide long-term protection against SARS-CoV. Thus, vaccines capable of eliciting humoral as well as cell-mediated immune responses are most promising.

Several national and international research groups are working on the development of vaccines to prevent and treat the 2019-nCoV/SARS-CoV-2, but effective vaccines are not available yet. Thus, there remains an imminent need for an effective therapeutic and/or prophylactic vaccine that can be developed and approved in a short period of time.

### SUMMARY OF THE INVENTION

In view of the current understanding of the novel corona virus and the worldwide epidemic caused by SARS-CoV-2, it is an object of the present invention to provide a novel oral DNA vaccine for prevention and/or the treatment of coronavirus disease 2019 (COVID-19) or a SARS-CoV-2 infection. The DNA vaccine according to the present invention comprises a Salmonella typhi Ty21a strain comprising a DNA molecule comprising a eukaryotic expression cassette encoding at least a COVID-19 coronavirus (SARS-CoV-2) spike (S) protein or a portion thereof. This vaccine is based on a live attenuated Salmonella typhi strain referred to as Salmonella typhi Ty21a that serves as a carrier and adjuvant for the DNA molecule encoding the immunogenic antigen for expression within the host cells. This Salmonella-based carrier comprising the DNA molecule encoding the antigen can be developed and produced in a short period of time at large scale and may be adapted to potential mutations occurring in the virus if required.

Furthermore, the live, attenuated S. typhi Ty21a strain used as a carrier is the active component of Typhoral L®, also known as Vivotif® (manufactured by Berna Biotech Ltd., a Crucell Company, Switzerland), the only licensed live oral vaccine against typhoid fever. This vaccine has been extensively tested and has proved to be safe regarding patient toxicity as well as transmission to third parties (Wandan et al., J. Infectious Diseases 1982, 145:292-295). The vaccine is licensed in more than 40 countries and has been used in millions of individuals including thousands of children for prophylactic vaccination against typhoid fever. It has an unparalleled safety track record. The carrier used in the DNA vaccine of the present invention is therefore suited for getting approval and the product on the market in a short period of time.

The DNA vaccine according to the present invention therefore has several advantages that makes it particularly suitable for the challenge of providing an effective vaccine against COVID-19 and/or SARS-CoV-2 infection.

Provided herein is a DNA vaccine comprising a Salmonella typhi Ty21a strain comprising a DNA molecule comprising a eukaryotic expression cassette encoding at least a COVID-19 coronavirus (SARS-CoV-2) spike (S) protein or a portion thereof. In certain embodiments the COVID-19 coronavirus (SARS-CoV-2) spike (S) protein or a portion thereof comprises (a) a SARS-CoV-2 full-length S protein; (b) a SARS-CoV-2 S protein ectodomain; (c) a SARS-CoV-2 S protein subunit S1; (d) a SARS-CoV-2 S protein receptor binding domain (RBD); or (d) at least 3 immune-dominant epitopes of SARS-CoV-2 S protein.

In one embodiment the COVID-19 coronavirus (SARS-CoV-2) spike (S) protein is a SARS-CoV-2 full-length S protein. The SARS-CoV-2 full-length S protein may comprise an amino acid sequence of SEQ ID NO: 1 or an amino acid sequence having at least 95% sequence identity with SEQ ID NO: 1. The SARS-CoV-2 full-length S protein may also be the full-length S protein of a variant of SARS-CoV-2, such as lineage B.1.1.7, B.1.351 or B.1.1.28 (renamed P.1). The SARS-CoV-2 full-length S protein may also be a prefusion-stabilized form of the SARS-CoV-2 full-length S protein, such as comprising two or more stabilizing mutations. In one embodiment the prefusion-stabilized form of the SARS-CoV-2 full-length S protein comprises two stabilizing mutations to proline corresponding to amino acid position K986 and V987 in the amino acid sequence of SEQ ID NO: 1.

In certain embodiments the COVID-19 coronavirus (SARS-CoV-2) spike (S) protein or a portion thereof comprises the SARS-CoV-2 S protein ectodomain. The SARS-CoV-2 S protein ectodomain has an amino acid sequence of amino acid residues 1-1208 of SEQ ID NO: 1 or an amino acid sequence having at least 95% sequence identity with amino acid residues 1-1208 of SEQ ID NO: 1. The SARS-CoV-2 S protein ectodomain may also be the S protein ectodomain of a variant of SARS-CoV-2, such as lineage B.1.1.7, B.1.351 or P.1. The SARS-CoV-2 S protein or a portion thereof may also comprise a prefusion-stabilized form of the SARS-CoV-2 S protein ectodomain comprising two or more stabilizing mutations. In one embodiment the prefusion-stabilized form of the SARS-CoV-2 S protein ectodomain comprises two stabilizing mutations to proline corresponding to amino acid position K986 and V987 in the amino acid sequence of amino acid residues 1 to 1208 of SEQ ID NO: 1.

In certain embodiments the SARS-CoV-2 S protein or a portion thereof has an amino acid sequence of SEQ ID NO: 1 or an amino acid sequence having at least 95% sequence identity with SEQ ID NO: 1, comprising two stabilizing mutations K986P and V987P. In certain alternative embodiments SARS-CoV-2 S protein or a portion thereof comprises an amino acid sequence of amino acid residues 1-1208 of SEQ ID NO: 1 or an amino acid sequence having at least 95% sequence identity with amino acid residues 1-1208 of SEQ ID NO: 1, comprising two stabilizing mutations K986P and V987P.

In certain embodiments the COVID-19 coronavirus (SARS-CoV-2) spike (S) protein or a portion thereof comprises the SARS-CoV-2 S protein subunit S1. The SARS-CoV-2 protein subunit S1 may comprise an amino acid sequence of amino acid residues 1-681 of SEQ ID NO: 1 or an amino acid sequence having at least 95% sequence identity with amino acid residues 1-681 of SEQ ID NO: 1. The SARS-CoV-2 S protein subunit S1 may also be the S protein subunit S1 of a variant of SARS-CoV-2, such as lineage B.1.1.7, B.1.351 or P.1

In certain embodiments the COVID-19 coronavirus (SARS-CoV-2) spike (S) protein or a portion thereof comprises the SARS-CoV-2 S protein receptor binding domain (RBD). The SARS-CoV-2 protein RBD may comprise an amino acid sequence of amino acid residues 319-541 of SEQ ID NO: 1 or an amino acid sequence having at least 95% sequence identity with amino acid residues 319-541 of SEQ ID NO: 1. The SARS-CoV-2 S protein RBD may also be the S protein RBD of a variant of SARS-CoV-2, such as lineage B.1.1.7, B.1.351 or P.1

The DNA vaccine according to the invention may comprise a DNA molecule encoding the SARS-CoV-2 S protein or a portion thereof and optionally further encoding another SARS-CoV-2 protein or a portion thereof, preferably a SARS-CoV-2 N protein. In certain embodiments the eukaryotic expression cassette encodes the SARS-CoV-2 S protein or a portion thereof and further encodes another SARS-CoV-2 protein or a portion thereof, such as a SARS-CoV-2 N protein or a portion thereof.

The DNA vaccine according to the invention may further comprise one or more pharmaceutically acceptable excipients. In certain embodiments the DNA vaccine is an oral dosage form, such as an enteric coated capsule, a lyophilized powder or a suspension. The DNA vaccine according to the invention may further comprising one or more adjuvants.

Also provided herein is the DNA vaccine according to the invention for use in the treatment and/or the prevention of coronavirus disease 2019 (COVID-19) or a SARS-CoV-2 infection.

Also provided herein is a method for treating and/or preventing coronavirus disease 2019 (COVID-19) or a SARS-CoV-2 infection comprising administering the DNA vaccine according to the invention to a patient in need thereof. In preferred embodiments the DNA vaccine is administered orally. In certain embodiments a single dose of the DNA vaccine comprises the Salmonella typhi Ty21a strain at about 1×10⁶ to about 1×10⁹ colony forming units (CFU), and/or the DNA vaccine is to be administered 2 to 4 times in one week for priming, optionally followed by at least one boosting dose. In one embodiment the DNA vaccine is to be administered 2 to 4 times within the first week, followed by one or more single dose boosting each at least 2 weeks later, preferably each at least 4 weeks later.

### SHORT DESCRIPTION OF THE FIGURES

FIG. 1: Amino acid sequence of SARS-CoV-2 Spike protein (SEQ ID NO: 1) with amino acid residues 1-1208 marked as underlined and residues K986, V987, R682G, R683S and R685S in bold.

FIG. 2: Plasmid map of pVAX10.SCV-1

FIG. 3: SARS-CoV-2 constructs for cloning into pVAX10, with the X indicating the presence of the domain in the order from N-terminal (left) to C-terminal (right), The following abbreviations are used; S FL (full-length S protein, SEQ ID NO: 1; *indicates signal domain (Met1-SER12 of SEQ ID NO: 1) replaced with that of invariant chain (Met1-Arg29 of SEQ ID NO: 15)), S ecto: (S protein ectodomain), S1 (S protein S1 subunit), RBD (receptor binding domain), T4 trimer (T4 fibritin trimerization motif), 3C3d (enhancer sequence comprising three copies of the C3d protein), 2A (2A peptide, such as T2a or P2a), Ubi. (ubiquitin), N (N protein), S2 (S protein S2 subunit) and SV40 DTS (SV40 DNA nuclear targeting sequence).

FIG. 4: Immune responses elicited by VXM-SCV-3 in healthy mice. The serum of vaccinated mice was analysed for antibodies against SARS-CoV spike protein (see Example 5). The assay background lies at 400 endpoint titer, as indicated by the dotted straight line.

FIG. 5: Immune responses elicited by VXM-SCV-30 in healthy mice. The serum of vaccinated mice was analysed for antibodies towards SARS-CoV spike protein (see Example 6). The assay background lies at 400 endpoint titer, as indicated by the straight line.

FIG. 6: Immune responses elicited by VXM-SCV-42 in healthy mice. The serum of vaccinated mice was analysed for antibodies towards SARS-CoV spike protein (see Example 7). The assay background lies at 400 endpoint titer, as indicated by the dotted straight line.

FIG. 7: Immune responses elicited by VXM-SCV-53 in healthy mice. The serum of vaccinated mice was analysed for antibodies towards SARS-CoV spike protein (see Example 8). The assay background lies at 400 endpoint titer, as indicated by the dotted straight line.

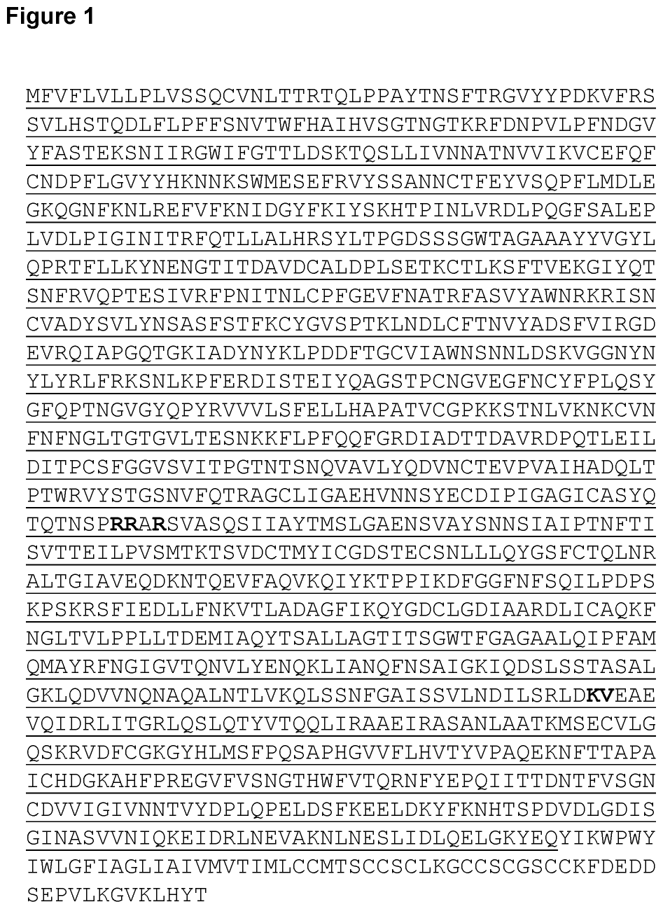

### DETAILED DESCRIPTION OF THE INVENTION

Provided herein is a DNA vaccine comprising a Salmonella typhi Ty21a strain comprising a DNA molecule comprising a eukaryotic expression cassette encoding at least a COVID-19 coronavirus (SARS-CoV-2) spike (S) protein or a portion thereof.

According to the invention, the Salmonella typhi Ty21a strain functions as the bacterial carrier of the DNA molecule comprising a eukaryotic expression cassette encoding at least a COVID-19 coronavirus (SARS-CoV-2) spike (S) protein or a portion thereof for the delivery of said DNA molecule into a target cell. Thus, the DNA molecule is delivered to a host cell and the S protein or a portion thereof is expressed by the host cell. The strain Salmonella typhi Ty21a is an attenuated Salmonella typhi strain and the DNA vaccine according to the invention comprises the live attenuated Salmonella typhi strain Salmonella typhi Ty21a.

In the context of the present invention, the term “attenuated” refers to a bacterial strain of reduced virulence compared to the parental bacterial strain, not harboring the attenuating mutation. Attenuated bacterial strains have preferably lost their virulence but retained their ability to induce protective immunity. Attenuation can be accomplished by deletion of various genes, including virulence, regulatory, and metabolic genes. Attenuated bacteria may be found naturally or they may be produced artificially in the laboratory, for example by adaptation to a new medium or cell culture or they may be produced by recombinant DNA technology. Administration of about 10¹¹ CFU of the attenuated strain of Salmonella according to the present invention preferably causes Salmonellosis in less than 5%, more preferably less than 1%, most preferably less than 1‰ of subjects.

The term “comprises” or “comprising” means “including, but not limited to”. The term is intended to be open-ended, to specify the presence of any stated features, elements, integers, steps or components, but not to preclude the presence or addition of one or more other features, elements, integers, steps, components or groups thereof. The term “comprising” thus includes the more restrictive terms “consisting of” and “essentially consisting of”. In one embodiment the term “comprising” may be individually replaced by the term “consisting of”. With regard to sequences the terms “having an amino acid sequence of” and “comprising an amino acid of” are used interchangeably and include the embodiment “consisting of the amino acid sequence of”. The term “a” as used herein may include the plural and hence includes, but is not limited, to “one”.

The term “SARS-CoV-2 S protein or a portion thereof” or “another SARS-CoV-2 protein or a portion thereof” as used herein refers to the SARS-CoV-2 S protein or an immunogenic portion thereof or another SARS-CoV-2 protein and an immunogenic portion thereof. An immunogenic portion of a protein may comprise one or more domain(s) of the immunogenic protein. However, it is also encompassed by the present invention that the immunogenic portion comprises only the immunogenic part of a domain, such as the receptor binding domain or the ectodomain. The term “immunogenic” as used herein refers to a part of protein that elicits an immune response, such as a B cell and/or T cell response.

A DNA molecule comprising at least one eukaryotic expression cassette may also be referred to as a recombinant DNA molecule, i.e. an engineered DNA construct, preferably composed of DNA pieces of different origin. The DNA molecule can be a linear nucleic acid or a circular nucleic acid. Preferably the DNA molecule is a plasmid, more preferably an expression plasmid. The plasmid may be generated by introducing an open reading frame encoding at least the SARS-CoV-2 S protein or a portion thereof into a eukaryotic expression cassette of a plasmid. A plasmid comprising a eukaryotic expression cassette may also be referred to as eukaryotic expression plasmid.

In the context of the present invention, the term “expression cassette” refers to a nucleic acid unit comprising at least one open reading frame (ORF) under the control of regulatory sequences controlling its expression. Preferably the expression cassette also comprises a transcription termination signal. Expression cassettes can preferably mediate transcription of the included open reading frame encoding at least the SARS-CoV-2 S protein or a portion thereof in a target cell. Eukaryotic expression cassettes typically comprise a promoter, at least one open reading frame and a transcription termination signal, which allow expression in a eukaryotic target cell.

Coronaviruses are positive-sense single-stranded RNA viruses belonging to the family Coronaviridae. These viruses mostly infect animals, including birds and mammals. In humans, coronaviruses typically cause mild respiratory infections. Since 2003 two highly pathogenic human Coronaviruses including Severe Acute Respiratory Syndrome Coronavirus (SARS-CoV) and Middle East Respiratory Syndrome Coronavirus (MERS-CoV) have led to global epidemics with high morbidity and mortality. Both endemics were caused by zoonotic coronaviruses that belong to the genus Betacoronavirus within Coronaviridae.

Like SARS-CoV and MERS-CoV, the new coronavirus SARS-CoV-2 belongs to the Betacoronavirus genus. The genome of SARS-CoV-2 has about 30 kilobase and encodes for multiple structural and non-structural proteins. The structural proteins include the spike (S) protein, the envelope (E) protein, the membrane (M) protein, and the nucleocapsid (N) protein. As reported by Zhou et al. (Cell Discovery (2020) 6:14) SARS-CoV-2 shares the highest nucleotide sequence identity with SARS-CoV (79.7%). Specifically, the envelope and nucleocapsid proteins of SARS-CoV-2 are two evolutionarily conserved regions, with sequence identities of 96% and 89.6%, respectively, compared to SARS-CoV. The spike protein was reported to exhibit the lowest sequence conservation (sequence identity of 77%) between SARS-CoV-2 and SARS-CoV, while the spike protein of SARS-CoV-2 only has 31.9% sequence identity with the spike protein of MERS-CoV. Several non-structural proteins were predicted for SARS-CoV-2 which are coded for by the open reading frames ORF lab, ORF 3a, ORF3b, ORF6, ORF 7a, ORF7b, ORFS, ORF9a, ORF9b, and ORF10 (Srinivasan et al. Viruses (2020) 12:360). In the meantime, several variants of SARS-CoV-2 were identified, for instance, the SARS-CoV-2 lineage B.1.1.7 first reported in the UK, the B.1.351 lineage first reported in South Africa and the B.1.1.28 subclade first reported in Brazil which was renamed as P.1 (Galloway et al., MMWR Morb Mortal Wkly Rep. 2021 Jan. 22; 70(3): 95-99). According to Galloway et al. these variants carry a constellation of genetic mutations, including in the S protein receptor-binding domain, which is essential for binding to the host cell angiotensin-converting enzyme-2 (ACE-2) receptor to facilitate virus entry. It seems that these variants spread more efficiently.

Various reports related to SARS-CoV suggest a protective role of both humoral and cell-mediated immune response. The S protein is the most exposed protein and antibody responses against the SARS-CoV S protein have been shown to protect from SARS-CoV infection in a mouse model. While being effective antibody responses may be short-lived. In contrast, T cell responses have been shown to provide long-term protection. In addition, multiple studies have shown that antibodies are generated against the N protein of SARS-CoV and by extension to SARS-CoV-2, the N protein is considered to be a highly immunogenic and abundantly expressed protein during infection. Further, of the structural proteins, T cell responses against the S and N proteins have been reported to be the most dominant and long-lasting (Ahmed et al. Viruses (2020) 12:254). The attenuated strain of Salmonella, Salmonella typhi Ty21a, is of the species Salmonella enterica. Attenuated derivatives of Salmonella enterica are attractive vehicles for the delivery of heterologous antigens to the mammalian immune system, since S. enterica strains can potentially be delivered via mucosal routes of immunization, i.e. orally or nasally, which offers advantages of simplicity and safety compared to parenteral administration. Furthermore, Salmonella strains elicit strong humoral and cellular immune responses at the level of both systemic and mucosal compartments. Batch preparation costs are low and formulations of live bacterial vaccines are highly stable. Attenuation can be accomplished by deletion of various genes, including virulence, regulatory, and metabolic genes.

Several Salmonella typhimurium strains attenuated by aro mutations have been shown to be safe and effective delivery vehicles for heterologous antigens in animal models.

The attenuated strain Salmonella typhi Ty21a has been shown to be safe and effective as a vaccine against typhoid fever and as a delivery vehicle for heterologous antigens for vaccination in humans, primarily for vaccination against tumor antigens and/or stroma antigens.

The live, attenuated S. typhi Ty21 a strain is the active component of Typhoral L®, also known as Vivotif® manufactured by Berna Biotech Ltd., a Crucell Company, Switzerland). It is currently the only licensed live oral vaccine against typhoid fever. This vaccine has been extensively tested and has proved to be safe regarding patient toxicity as well as transmission to third parties (Wandan et al., J. Infectious Diseases 1982, 145:292-295). The vaccine is licensed in more than 40 countries and has been used in millions of individuals including thousands of children for prophylactic vaccination against typhoid fever. The Marketing Authorization number of Typhoral L® is PL 15747/0001 dated 16 Dec. 1996. One dose of vaccine contains at least 2×10⁹ viable S. typhi Ty21a colony forming units and at least 5×10⁹ non-viable S. typhi Ty21a cells.

This well-tolerated, live oral vaccine against typhoid fever was derived by chemical mutagenesis of the wild-type virulent bacterial isolate S. typhi Ty2 and harbors a loss-of-function mutation in the galE gene resulting in its inability to metabolize galactose. The attenuated bacterial strain is also not able to reduce sulfate to sulfide which differentiates it from the wild-type Salmonella typhi Ty2 strain. With regard to its serological characteristics, the Salmonella typhi Ty21a strain contains the 09-antigen which is a polysaccharide of the outer membrane of the bacteria and lacks the 05-antigen which is in turn a characteristic component of Salmonella typhimurium. This serological characteristic supports the rationale for including the respective test in a panel of identity tests for batch release.

SARS-CoV-2 S protein is a glycoprotein with 66 N-linked glycosylation sites per trimer. The protein also comprises O-linked glycans at residues S673, T678 and S686. Furthermore, the S protein contains two functional domains: a receptor binding domain, and a second domain which contains sequences that mediate fusion of the viral and cell membranes. The S glycoprotein must be cleaved by cell proteases to enable exposure of the fusion sequences and hence is needed for cell entry. Protein sequence of the S glycoprotein of SARS-CoV-2 reveals the presence of a furin cleavage sequence (PRRARSIV) at residues 681-687 due to an insertion of the sequence PRRA. Because furin proteases are abundant in the respiratory tract, it is possible that SARS-CoV-2 S glycoprotein is cleaved upon exit from epithelial cells and consequently can efficiently infect other cells.

The expression cassette used for the DNA vaccine according to the invention is a eukaryotic expression cassette. In the context of the present invention, the term “eukaryotic expression cassette” refers to an expression cassette which allows for expression of the open reading frame in a eukaryotic cell. It has been shown that the amount of heterologous antigen required to induce an adequate immune response may be toxic for the bacterium and may result in cell death, over-attenuation or loss of expression of the heterologous antigen. Using a eukaryotic expression cassette that is not expressed in the bacterial vector but only in the target cell may overcome this toxicity problem and the protein expressed typically exhibits a eukaryotic glycosylation pattern.

A eukaryotic expression cassette comprises regulatory sequences that are able to control the expression of an open reading frame in a eukaryotic cell, preferably a promoter and a polyadenylation signal. Promoters and polyadenylation signals included in the recombinant DNA molecules comprised by the attenuated strain of Salmonella of the present invention are preferably selected to be functional within the cells of the subject to be immunized. Examples of suitable promoters, especially for the production of a DNA vaccine for humans, include but are not limited to promoters from Cytomegalovirus (CMV), such as the strong CMV immediate early promoter, Simian Virus 40 (SV40), Mouse Mammary Tumor Virus (MMTV), Human Immunodeficiency Virus (HIV), such as the HIV Long Terminal Repeat (LTR) promoter, Moloney virus, Epstein Barr Virus (EBV), and from Rous Sarcoma Virus (RSV), the synthetic CAG promoter composed of the CMV early enhancer element, the promoter, the first exon and the first intron of chicken beta-actin gene and the splice acceptor of the rabbit beta globin gene, as well as promoters from human genes such as human actin, human myosin, human hemoglobin, human muscle creatine, and human metallothionein. In a particular embodiment, the eukaryotic expression cassette contains the CMV promoter. In the context of the present invention, the term “CMV promoter” refers to the strong immediate-early cytomegalovirus promoter.

Examples of suitable polyadenylation signals, especially for the production of a DNA vaccine for humans, include but are not limited to the bovine growth hormone (BGH) polyadenylation site, SV40 polyadenylation signals and LTR polyadenylation signals. In a particular embodiment, the eukaryotic expression cassette included in the recombinant DNA molecule comprised by the attenuated strain of Salmonella of the present invention comprises the BGH polyadenylation site.

In addition to the regulatory elements required for expression of the heterologous SARS-CoV-2 S protein or a portion thereof, like a promoter and a polyadenylation signal, other elements can also be included in the recombinant DNA molecule. Such additional elements include enhancers. The enhancer can be, for example, the enhancer of human actin, human myosin, human hemoglobin, human muscle creatine and viral enhancers such as those from CMV, RSV and EBV.

In the context of the present invention it is generally advantageous to use a gene (or open reading frame) encoding the SARS-CoV-2 S protein or a portion thereof (as well as an optional further SARS-CoV-2 protein or a portion thereof, such as the SARS-CoV-2 N protein or a portion thereof) that it codon-optimized for mammalian expression, particularly for human expression. Thus, in certain embodiments the eukaryotic expression cassette comprises at least a codon-optimized sequence encoding COVID-19 coronavirus (SARS-CoV-2) spike (S) protein or a portion thereof.

The COVID-19 coronavirus (SARS-CoV-2) spike (S) protein or a portion thereof encoded by the DNA vaccine according to the invention comprises without being limited thereto (a) a SARS-CoV-2 full-length S protein; (b) a SARS-CoV-2 S protein ectodomain; (c) a SARS-CoV-2 protein subunit S1; (d) a SARS-CoV-2 receptor binding domain (RBD) or (e) at least 3 immune-dominant epitopes of SARS-CoV-2 S protein.

In certain embodiments the COVID-19 coronavirus (SARS-CoV-2) spike (S) protein is a SARS-CoV-2 full-length S protein. The SARS-CoV-2 full-length S protein may comprise an amino acid sequence of SEQ ID NO: 1 or an amino acid sequence having at least 95% sequence identity with SEQ ID NO: 1. In a preferred embodiment the SARS-CoV-2 full-length S protein has an amino acid sequence having at least 96%, at least 97%, at least 98%, or at least 99% sequence identity with SEQ ID NO: 1. In one embodiment the SARS-CoV-2 full-length S protein has an amino acid sequence having at least 98% to 100% sequence identity with SEQ ID NO: 1. In a specific embodiment the COVID-19 coronavirus (SARS-CoV-2) spike (S) protein is a SARS-CoV-2 full-length S protein consisting of an amino acid sequence of SEQ ID NO: 1 or an amino acid sequence having at least 95% sequence identity with SEQ ID NO: 1. The amino acid sequence of SEQ ID NO: 1 has the GenBank accession number MN 908947 and has been published by Wu et al. (Nature 2020, 579: 265-269). In a specific embodiment the SARS-CoV-2 full-length S protein may also be the full-length S protein of a variant of SARS-CoV-2, such as lineage B.1.1.7, B.1.351 or P.1.

We compared different S protein sequences of SARS-CoV-2 available at GenBank in an alignment of the sequences of the GenBank accession numbers (protein-id): MN_908947 (QHD434616.1), MN_988668 (QHQ62107.1), NC_045512 (YP_009724390.1), MN_938384.1 (QHN73795.1), MN_975262.1 (QHN73810.1), MN_985325.1 (QHQ60594.1), MN_988713.1 (QHQ62877.1), MN_994467.1 (QHQ71963.1), MN_994468.1 (QHQ71973.1), and MN997409.1 (QHQ82464.1) and found no differences. However, minor variations have previously been reported in the SARS-CoV-2 S protein. For example the following substitutions have been described by Wrapp et al. (Science, 2020, 367: 1260-1263) in clinical isolates F321, H49Y, S247R, N354D, D364Y, V367F, D614G, V1129L and E1262G. Moreover the substitutions H49Y and V860Q have been reported by Wang et al. (J. Med. Virol. Mar. 13, 2020: 1-8). Further homology analysis of the published SARS-CoV-2 sequences by the same authors revealed a nucleotide homology of the S protein of 99.82% to 100% and an amino acid homology of the S protein of 99.53% to 100%. The identified variants B.1.1.7, B.1.351 and P.1 carry several mutations. The B.1.1.7 variant S protein has the deletions 69-70 HV and 144 Y and the following mutations: N501Y, A570D, D614G, P681H, T7611, S982A, D1118H. The variant B.1.351 carries the following mutations in the S protein: K417N, E484K, N501Y, D614G and A701V. The P.1 variant carries a L18F, T2ON, P26S, D138Y, R1905, K417T, E484K, N501Y, D614G, H655Y and T10271 mutation in the S protein (Galloway et al., MMWR Morb Mortal Wkly Rep. 2021 Jan. 22; 70(3): 95-99). However, further substitutions or variants may occur or be identified over time.

The SARS-CoV-2 full-length S protein may also be a prefusion-stabilized form of the SARS-CoV-2 full-length S protein, such as comprising two or more stabilizing mutations. In certain embodiments the prefusion-stabilized form of the SARS-CoV-2 full-length S protein comprises two stabilizing mutations to proline corresponding to amino acid position K986 and V987 in the amino acid sequence of SEQ ID NO: 1.

Prefusion-stabilized forms of SARS-CoV-2 S protein have been described by Wrapp et al. (Science, 2020, 367: 1260-1263) by adding two stabilizing proline mutations at residues 986 and 987 in the C-terminal S2 fusion machinery using a previously stabilizing strategy that proved effective for other betacoronavirus S proteins. Furthermore, Wrapp et al. (Science, 2020, 367: 1260-1263) described a “GSAS” mutation in the furin cleavage site at residues 682-685, replacing the RRAR sequence at this position. Both these mutations stabilize the protein and hence prevent fusion. This may not only improve stability and expression of the S protein, but also improve safety by preventing cell fusion. In certain embodiments the prefusion-stabilized form of the SARS-CoV-2 full-length S protein comprises two stabilizing mutations to proline corresponding to amino acid position K986 and V987 in the amino acid sequence of SEQ ID NO: 1 and/or a mutation of the furin cleavage sequence (PRRARSIV) corresponding to residues 681-687 of SEQ ID NO: 1, such as a R682G, R683S and R685S mutation. Preferably the SARS-CoV-2 full-length S protein has an amino acid sequence of SEQ ID NO: 1 or an amino acid sequence having at least 95% sequence identity with SEQ ID NO: 1, further comprising two stabilizing mutations K986P and V987P; or furin cleavage sequence mutations R682G, R683S and R685S, or two stabilizing mutations K986P and V987P and furin cleavage sequence mutations R682G, R683S and R685S. Alternatively amino acids of the furin cleavage sequence may be deleted such as amino acids 680-683. Thus, in one embodiment the SARS-CoV-2 full-length S protein has an amino acid sequence of SEQ ID NO: 1 or an amino acid sequence having at least 95% sequence identity with SEQ ID NO: 1, further comprising a deletion in the furin cleavage sequence, such as a deletion comprising or consisting of amino acids 5680-R683. Other amino acid substitutions or amino acid deletions resulting in a pre-fusion stabilized form of the S protein may also be employed.

In certain embodiments the COVID-19 coronavirus (SARS-CoV-2) spike (S) protein or a portion thereof comprises the SARS-CoV-2 S protein ectodomain. The term “ectodomain” refers to the extracellular portion of the transmembrane protein SARS-CoV-2 S protein, i.e., lacking the transmembrane domain and the cytoplasmic domain. The ectodomain comprises the membrane distal subunit S1 comprising the receptor binding domain and the membrane proximate subunit S2. The SARS-CoV-2 S protein ectodomain comprises an amino acid sequence of amino acid residues 1-1208 of SEQ ID NO: 1 or an amino acid sequence having at least 95% sequence identity with amino acid residues 1-1208 of SEQ ID NO: 1. However the SARS-CoV-2 S protein ectodomain as used herein may be a sequence corresponding at least to amino acid residues 1 to 1208 of SEQ ID NO: 1 or may be slightly longer, such as up to the N-terminal 1213 amino acid residues of SEQ ID NO: 1 or a sequence having at least 95% sequence identity with amino acid residues 1-1213 of SEQ ID NO: 1. In a preferred embodiment the SARS-CoV-2 S protein or a portion thereof comprises the SARS-CoV-2 S protein ectodomain having an amino acid sequence having at least 96%, at least 97%, at least 98%, or at least 99% sequence identity with the sequence of amino acid residues 1-1208 of SEQ ID NO: 1. In one embodiment the SARS-CoV-2 S protein ectodomain has an amino acid sequence having at least 98% to 100% sequence identity with amino acid residues 1-1208 of SEQ ID NO: 1. In a specific embodiments the COVID-19 coronavirus (SARS-CoV-2) spike (S) protein or a portion thereof is the SARS-CoV-2 S protein ectodomain having or consisting of an amino acid sequence of amino acid residues 1-1208 of SEQ ID NO: 1 or an amino acid sequence having at least 95% sequence identity with amino acid residues 1-1208 of SEQ ID NO: 1. In a further specific embodiment the SARS-CoV-2 S protein ectodomain may also be the S protein ectodomain of a variant of SARS-CoV-2, such as lineage B.1.1.7, B.1.351 or P.1.

The SARS-CoV-2 S protein or a portion thereof may also comprise a prefusion-stabilized form of the SARS-CoV-2 S protein ectodomain comprising two or more stabilizing mutations. In one embodiment the prefusion-stabilized form of the SARS-CoV-2 S protein ectodomain comprises two stabilizing mutations to proline corresponding to amino acid position K986 and V987 in the amino acid sequence of amino acid residues 1 to 1208 of SEQ ID NO: 1.

In certain embodiments the SARS-CoV-2 S protein or a portion thereof comprises an amino acid sequence of amino acid residues 1-1208 of SEQ ID NO: 1 or an amino acid sequence having at least 95% sequence identity with amino acid residues 1-1208 of SEQ ID NO: 1, further comprising two stabilizing mutations K986P and V987P.

In certain embodiments the prefusion-stabilized form of the SARS-CoV-2 S protein ectodomain comprises two stabilizing mutations to proline corresponding to amino acid position K986 and V987 in the amino acid sequence of amino acid residues 1-1208 of SEQ ID NO: 1 and/or a mutation of the furin cleavage sequence (PRRARSIV) corresponding to residues 681-687 of the amino acid sequence of amino acid residues 1-1208 of SEQ ID NO: 1, such as a R682G, R683S and R685S mutation. Preferably the SARS-CoV-2 S protein ectodomain has an amino acid sequence of amino acid residues 1-1208 of SEQ ID NO: 1 or an amino acid sequence having at least 95% sequence identity with the amino acid sequence of amino acid residues 1-1208 of SEQ ID NO: 1, comprising two stabilizing mutations K986P and V987P; or furin cleavage sequence mutations R682G, R683S and R685S, or two stabilizing mutations K986P and V987P and furin cleavage sequence mutations R682G, R683S and R685S. Alternatively amino acids of the furin cleavage sequence may be deleted such as amino acids 680-683. Thus, in one embodiment the SARS-CoV-2 full-length S protein has an amino acid sequence of SEQ ID NO: 1 or an amino acid sequence having at least 95% sequence identity with SEQ ID NO: 1, comprising a deletion in the furin cleavage sequence, such as a deletion comprising or consisting of amino acids 5680-R683. Other amino acid substitutions or amino acid deletions resulting in a pre-fusion stabilized form of the S protein ectodomain may also be employed.

The SARS-CoV-2 ectodomain may further comprise a fusion domain for stabilization and/or improved expression and/or improved secretion. The fusion domain may also be a trimerization domain, such as a C-terminal T4 fibritin timerization motif. The trimerization domain of the bacteriophage T4 fibritin, termed “foldon”, has the amino acid sequence GYIPEAPRDGQAYVRKDGEVVVLLSTFL (SEQ ID NO: 10) corresponding to amino acid residues aa 457-483 of the fibritin protein.

The sequence encoding the SARS-CoV-2 S protein or a portion thereof preferably comprises a signaling sequence encoding a signaling peptide. The signaling peptide of the SARS-CoV-2 S protein has for example the amino acid sequence: MFVFLVLLPLVSSQC (SEQ ID NO: 3) corresponding to amino acid residues 1-15 of SEQ ID NO: 1 or an equivalent functional signaling peptide having at least 80% sequence identity, preferably at least 90% sequence identity, with the amino acid sequence of SEQ ID NO: 3. In one embodiment the signaling peptide of the SARS-CoV-2 S protein the signal peptide of the invariant chain, wherein in a preferred embodiment amino acid residues 1-12 of SEQ ID NO: 1 is replaced with amino acid residues 1-29 of SEQ ID NO: 15.

In certain embodiments the COVID-19 coronavirus (SARS-CoV-2) spike (S) protein or a portion thereof comprises the SARS-CoV-2 S protein subunit S1. The SARS-CoV-2 S protein subunit S1 comprises an amino acid sequence of amino acid residues 1-681 of SEQ ID NO: 1 or an amino acid sequence having at least 95% sequence identity with amino acid residues 1-681 of SEQ ID NO: 1. In a preferred embodiment the SARS-CoV-2 S protein or a portion thereof comprises the SARS-CoV-2 S protein subunit S1 having an amino acid sequence having at least 96%, at least 97%, at least 98%, or at least 99% sequence identity with the sequence of amino acid residues 1-681 of SEQ ID NO: 1. In one embodiment the SARS-CoV-2 S protein subunit S1 has an amino acid sequence having at least 98% to 100% sequence identity with amino acid residues 1-681 of SEQ ID NO: 1. In a specific embodiments the COVID-19 coronavirus (SARS-CoV-2) spike (S) protein or a portion thereof is the SARS-CoV-2 S protein subunit S1 having or consisting of an amino acid sequence of amino acid residues 1-681 of SEQ ID NO: 1 or an amino acid sequence having at least 95% sequence identity with amino acid residues 1-681 of SEQ ID NO: 1. In a further specific embodiment the SARS-CoV-2 S protein subunit S1 may also be the S protein subunit S1 of a variant of SARS-CoV-2, such as lineage B.1.1.7, B.1.351 or P.1.

In certain embodiments the COVID-19 coronavirus (SARS-CoV-2) spike (S) protein or a portion thereof comprises the SARS-CoV-2 S protein receptor binding domain (RBD). The SARS-CoV-2 S protein RBD comprises an amino acid sequence of amino acid residues 319-541 of SEQ ID NO: 1 or an amino acid sequence having at least 95% sequence identity with amino acid residues 319-541 of SEQ ID NO: 1. In a preferred embodiment the SARS-CoV-2 S protein or a portion thereof comprises the SARS-CoV-2 S protein RBD having an amino acid sequence having at least 96%, at least 97%, at least 98%, or at least 99% sequence identity with the sequence of amino acid residues 319-541 of SEQ ID NO: 1. In one embodiment the SARS-CoV-2 S protein RBD has an amino acid sequence having at least 98% to 100% sequence identity with amino acid residues 319-541 of SEQ ID NO: 1. In a specific embodiments the COVID-19 coronavirus (SARS-CoV-2) spike (S) protein or a portion thereof is the SARS-CoV-2 S protein RBD having or consisting of an amino acid sequence of amino acid residues 319-541 of SEQ ID NO: 1 or an amino acid sequence having at least 95% sequence identity with amino acid residues 319-541 of SEQ ID NO: 1. In a specific embodiment the SARS-CoV-2 S protein RBD may also be the S protein RBD of a variant of SARS-CoV-2, such as lineage B.1.1.7, B.1.351 or P.1.

One advantage of using the SARS-CoV-2 full-length S protein, the SARS-CoV-2 S protein ectodomain, the SARS-CoV-2 protein subunit S1 or the SARS-CoV-2 RBD is that it provides for a polyclonal humoral immune response (including a neutralizing antibody response) maintaining efficacy over mutating SARS-CoV-2 and that the humoral as well as the cellular immune response is not MHC restricted and hence limited to patients with certain HLA type.

In the context of the present invention, the term “at least 95% sequence identity with” refers to a protein that may differ in the amino acid sequence and/or the nucleic acid sequence encoding the amino acid sequence of the reference sequence, such as the amino acid sequence of SEQ ID NO: 1 or the amino acid sequence of amino acid residues 1-1208, amino acid residues 1-681 or amino acid residues 319-541 of SEQ ID NO: 1 (also referred to as the corresponding portion thereof). The S protein or the portion thereof may be of natural origin, e.g. a mutant version or a variation of the S protein of SARS-CoV-2 having the amino acid sequence of SEQ ID NO: 1 or an engineered protein, e.g. an engineered glycoprotein derivative, which has been modified by introducing site directed mutations or cloning, or a combination thereof. It is known that the usage of codons is different between species. Thus, when expressing a heterologous protein in a target cell, it may be necessary, or at least helpful, to adapt the nucleic acid sequence to the codon usage of the target cell. Methods for designing and constructing derivatives of a given protein are well known to the person skill in the art. Adapting the nucleic acid sequence to the codon usage of the target cell is also known as codon-optimization.

The S protein or a portion thereof that shares at least about 95% sequence identity with the amino acid sequence of SEQ ID NO: 1 or the corresponding portion thereof may contain one or more mutations comprising an addition, a deletion and/or a substitution of one or more amino acids. According to the teaching of the present invention, said deleted, added and/or substituted amino acids may be consecutive amino acids or may be interspersed over the length of the amino acid sequence of the S protein or the portion thereof that shares at least about 95% sequence identity with the amino acid sequence of SEQ ID NO: 1 or the corresponding portion thereof. According to the teaching of the present invention, any number of amino acids may be added, deleted, and/or substitutes, as long as the amino acid sequence identity with the amino acid sequence of SEQ ID NO: 1 or the corresponding portion thereof is at least about 95%. In particular embodiments, the sequence identity of the amino acid sequence of the S protein or a portion thereof with the amino acid sequence of SEQ ID NO: 1 or the corresponding portion thereof is at least 95%, at least 96%, at least 97%, at least 98%, or preferably at least 99%. All percentages are in relation to the amino acid sequence of SEQ ID NO: 1 or the corresponding portion thereof (such as amino acid residues 1-1208, amino acid residues 1-681 or amino acid residues 329-541). Methods and algorithms for determining sequence identity including the comparison of a parental protein and its derivative having deletions, additions and/or substitutions relative to a parental sequence, are well known to the practitioner of ordinary skill in the art. On the DNA level, the nucleic acid sequences encoding the S protein or a portion thereof that shares at least about 95% sequence identity with the amino acid sequence of SEQ ID NO: 1 may differ to a larger extent due to the degeneracy of the genetic code and the optional codon-optimization.

According to the invention, the DNA vaccine may comprise in certain embodiments a Salmonella typhi Ty21a strain comprising a DNA molecule comprising a eukaryotic expression cassette encoding from N-terminal to C-terminal at least a SARS-CoV-2 S protein or a portion thereof and an enhancer sequence, such as a complement peptide sequence, more preferably three copies of complement protein C3d (SEQ ID NO: 4) preferably each of the three C3d separated by a GS linker (3C3d; SEQ ID NO: 5). Such sequences have been described to enhance humoral immune responses, particularly eliciting a stronger antibody response. In case the SARS-CoV-2 S protein or a portion thereof comprises the SARS-CoV-2 S protein ectodomain, the SARS-CoV-2 S protein subunit S1 or the SARS-CoV-2 S protein RBD, the eukaryotic expression cassette may further encode a trimerization domain, such as a C-terminal T4 fibritin trimerization motif (SEQ ID NO: 10), preferably fused to the SARS-CoV-2 S protein portion. Thus, in certain embodiments, the DNA vaccine may also comprise a Salmonella typhi Ty21a strain comprising a DNA molecule comprising a eukaryotic expression cassette encoding from N-terminal to C-terminal at least the SARS-CoV-2 protein or a portion thereof comprising the SARS-CoV-2 S protein ectodomain, the SARS-CoV-2 S protein subunit S1 or the SARS-CoV-2 S protein RBD (preferably the SARS-CoV-2 S protein ectodomain), a trimerization domain and optionally an enhancer sequence, such as a complement peptide sequence.

Exemplary enhancers sequences such as ubiquitin peptide sequences or complement peptide sequences to promote presentation of antigens in MHC class I or II molecules, respectively, are known in the art. Plasmid vectors encoding MHC class I antigens and ubiquitin peptides delivered by Salmonella typhimurium to murine have demonstrated enhanced antigen-specific T cell responses and tumour control in a B16 tumour challenge model (Xiang et al, PNAS, 2000). Antibody responses to B cell epitopes encoded by DNA vectors have been shown to be enhanced by introduction of three copies of peptides of complement protein C3d, which binds to the CR2 (CD21) receptor found on B cells and follicular dendritic cells to enhance antigen-specific B cell activation (Moveseyan, J Neuroimmunol, 2008; Yang, Virus Res, 2010; Hou, Virology J, 2019). Thus, in order to enhance B cell responses, complement peptides sequences such as three copies of complement protein C3d (KFLTTAKDKNRWEDPGKQLYNVEATSYA; SEQ ID NO: 4) may be added C-terminally to the sequence encoding the SARS-CoV-2 S protein or a portion thereof. Preferably the three 28 amino acid peptides are separated by a GS linker, such as GS(G4S)₂GS as in SEQ ID NO: 5 (3C3d). Further, to improve nuclear import of the DNA molecule (such as a plasmid) comprising the eukaryotic expression cassette encoding at least a SARS-CoV-2 S protein or a portion thereof from the cytoplasm, the DNA molecule may further comprise a DNA nuclear targeting sequence, such as one or more copies of the SV40 DNA nuclear targeting sequence (DTS; SEQ ID NO: 16), preferably two or more copies of the DTS.

The DNA vaccine according to the invention may further encode another SARS-CoV-2 protein or a portion thereof, preferably a SARS-CoV-2 N protein or a portion thereof. In preferred embodiments the SARS-CoV-2 N protein or a portion thereof comprises the sequence of SEQ ID NO: 8 or a portion thereof or a sequence having at least 95% sequence identity with SEQ ID NO: 8 or a corresponding portion thereof. Preferably the SARS-CoV-2 N protein or a portion thereof has an amino acid sequence having at least 96%, at least 97%, at least 98%, or at least 99% sequence identity with the sequence of SEQ ID NO: 8. In one embodiment the SARS-CoV-2 N protein or a portion thereof has an amino acid sequence having at least 98% to 100% sequence identity with the sequence of SEQ ID NO: 8 or the corresponding portion thereof. In a further embodiment, the SARS-CoV-2 N protein or a portion thereof may also have the amino acid sequence of a variant of SARS-CoV-2, such as lineage B.1.1.7, B.1.351 or P.1.

The another SARS-CoV 2 protein or a portion thereof may be expressed by a further DNA vaccine comprising a Salmonella typhi Ty21a strain comprising a DNA molecule comprising a eukaryotic expression cassette encoding at least a COVID-19 coronavirus (SARS-CoV-2) protein other than the spike (S) protein or a portion thereof. The two DNA vaccines may be co-administered to induce an immune response against the SARS-CoV-2 S protein and the another SARS-CoV-2 protein. Alternatively the another SARS-CoV 2 protein or a portion thereof may be expressed by the DNA vaccine according to the invention further comprising a second DNA molecule encoding the another SARS-CoV-2 protein. Thus, the DNA vaccine comprises a Salmonella typhi Ty21a strain comprising a first DNA molecule comprising a eukaryotic expression cassette encoding at least a COVID-19 coronavirus (SARS-CoV-2) protein spike (S) protein or a portion thereof and a second DNA molecule comprising a eukaryotic expression cassette encoding at least a COVID-19 coronavirus (SARS-CoV-2) protein other than the spike (S) protein or a portion thereof. Preferably the first and the second DNA molecules are plasmids, preferably expression plasmids. More preferably the plasmids have the same vector backbone, such as a pVAX10 backbone. It is also contemplated that the another SARS-CoV-2 protein or a portion thereof is expressed by the same DNA molecule comprising a first expression cassette encoding the SARS-CoV-2 S protein or a portion thereof and a second expression cassette encoding another SARS-CoV-2 protein or a portion thereof. All these embodiments may be freely combined with the embodiments referred to previously, particularly further defining the expression cassette encoding at least the SARS-CoV-2 S protein or a portion thereof optionally comprising an enhancer sequence and/or a trimerization domain.

It is further contemplated that the DNA molecule comprises a eukaryotic expression cassette encoding the SARS-CoV-2 S protein or a portion thereof and the another SARS-CoV-2 protein or a portion thereof. Thus, in certain embodiments the DNA vaccine comprises a Salmonella typhi Ty21a strain comprising a DNA molecule comprising a eukaryotic expression cassette encoding at least a COVID-19 coronavirus (SARS-CoV-2) spike (S) protein or a portion thereof and another COVID-19 coronavirus (SARS-CoV-2) protein (structural or non-structural). Preferably the SARS-CoV-2 S protein or a portion thereof is N-terminally expressed and the another SARS-CoV-2 protein or a portion thereof is C-terminally expressed. The following embodiments may be freely combined with the embodiments referred to previously, particularly further defining the expression cassette encoding at least the SARS-CoV-2 S protein or a portion thereof optionally comprising an enhancer sequence and/or a trimerization domain. In a preferred embodiment the DNA vaccine comprises a Salmonella typhi Ty21a strain comprising a DNA molecule comprising a eukaryotic expression cassette encoding at least a COVID-19 coronavirus (SARS-CoV-2) spike (S) protein or a portion thereof and a COVID-19 coronavirus (SARS-CoV-2) N protein or a portion thereof. The SARS-CoV-2 N protein or a portion thereof may comprise the sequence of SEQ ID NO: 8 or a portion thereof or a sequence having at least 95% sequence identity with SEQ ID NO: 8 or a corresponding portion thereof. Preferably the SARS-CoV-2 N protein or a portion thereof has an amino acid sequence having at least 96%, at least 97%, at least 98%, or at least 99% sequence identity with the sequence of SEQ ID NO: 8. In one embodiment the SARS-CoV-2 N protein or a portion thereof has an amino acid sequence having at least 98% to 100% sequence identity with the sequence of SEQ ID NO: 8 or the corresponding portion thereof. In one embodiment the SARS-CoV-2 N protein or a portion thereof may also have the amino acid sequence of a variant of SARS-CoV-2, such as lineage B.1.1.7, B.1.351 or P.1. The SARS-CoV-2 S protein or a portion thereof may be linked to the another SARS-CoV-2 protein via a 2A self-cleaving peptide (2A peptide) or an internal ribosomal entry site (IRES), preferably a 2A peptide. Examples of 2A peptides are P2a with the amino acid sequence of GSGATNFSLLKQAGDVEENPGP (SEQ ID NO: 6) or T2a with the amino acid sequence of GSGEGRGSLLTCGDVEENPGP (SEQ ID NO: 7).

According to the invention, the DNA vaccine may comprise a Salmonella typhi Ty21a strain comprising a DNA molecule comprising a eukaryotic expression cassette encoding from N-terminal to C-terminal at least a SARS-CoV-2 S protein or a portion thereof, a 2A peptide or an IRES sequence and another SARS-CoV-2 protein or a portion thereof, preferably a SARS-CoV-2 N protein or a portion thereof. The another SARS-CoV-2 protein or a portion thereof may further be followed by a SARS-CoV-2 protein subunit S2, particularly if the SARS-CoV-2 S protein or a portion thereof is the SARS-CoV-2 protein subunit S1. In certain embodiments, the SARS-CoV-2 protein subunit S2 comprises amino acid residues 686-1208 of SEQ ID NO: 1 or a sequence having at least 95% identity with amino acid residues 686-1208 of SEQ ID NO:1. In one embodiment subunit S2 comprises amino acid residues 686-1273 of SEQ ID NO: 1 or a sequence having at least 95% identity with amino acid residues 686-1273 of SEQ ID NO: 1.

The another SARS-CoV-2 protein or a portion thereof may further be preceded by an enhancer sequence, such as an ubiquitin sequence. Ubiquitin is conserved between mouse and human and has the amino acid sequence MQI FVKTLTGKTITLEVEPSDTI ENVKAKIQDKEGI PPDQQRLI FAGKQLEDGRTLSDYNIQKE STLHLVLRLRG (SEQ ID NO: 9). Without being bound by theory, a N-terminal ubiquitin sequence may enhance T cell responses of antigens. Thus, also contemplated is a DNA vaccine comprising a Salmonella typhi Ty21a strain comprising a DNA molecule comprising a eukaryotic expression cassette encoding from N-terminal to C-terminal at least a SARS-CoV-2 S protein or a portion thereof, a 2A peptide or an IRES sequence, an ubiquitin sequence and another SARS-CoV-2 protein or a portion thereof, preferably a SARS-CoV-2 N protein or a portion thereof, optionally followed by the SARS-CoV-2 protein subunit S2.

The N protein is considered to mainly elicit a T cell response. Plasmid vectors encoding MHC class I antigens and ubiquitin peptides delivered by Salmonella typhimurium to murine have demonstrated enhanced antigen-specific T cell responses and tumour control in a B16 tumour challenge model (Xiang et al, PNAS, 2000). Thus, T cell enhancing sequences may be fused, preferably N-terminally, to the another SARS-CoV-2 protein or a portion thereof, such as the SARS-CoV-2 N protein or a portion thereof.

The term “2A self-cleaving peptides”, “2A cleavage site” or “2A peptides” are used synonymously herein and refer to a class of 18-22 aa-long peptides, which can induce the cleaving of the recombinant protein in a cell. 2A peptides are originally found in the 2A region in a viral genome of virus and have been adopted as tool to express polypeptides in one expression cassette. The 2A-peptide-mediated cleavage occurs after the translation and the cleavage is trigged by breaking of peptide bond between the Proline (P) and Glycine (G) in C-terminal of 2A peptide. Sequences encoding 2A peptide linker are known in the art, such as provided in SEQ ID NOs: 6 or 7.

The term “internal ribosome entry site”, abbreviated IRES, as used herein is an RNA element that allows for translation initiation in a cap-independent manner and hence translation in an mRNA comprising an IRES sequences is also initiated at the IRES sequence.

In another embodiment the DNA vaccine comprising a Salmonella typhi Ty21a strain comprising a DNA molecule comprising a eukaryotic expression cassette encoding at least a COVID-19 coronavirus (SARS-CoV-2) spike (S) protein or a portion thereof, wherein the COVID-19 coronavirus (SARS-CoV-2) spike (S) protein or a portion thereof comprises at least 3 immune-dominant epitopes of SARS-CoV-2 S protein. In one embodiment the expression cassette encodes at least 3 immune-dominant epitopes of SARS-CoV-2 S protein and an enhancer sequence, such as a complement peptide sequence as described above.

The term “at least 3 immune-dominant epitopes of SARS-CoV-2 S protein” as used herein refers to one polypeptide or more than one polypeptide comprising together 3 or more immune-dominant epitopes of SARS-CoV-2 S protein. Whether the three or more immune-dominant epitopes of SARS-CoV-2 S protein are part of the same or different polypeptides is not relevant. The three or more immune-dominant epitopes of SARS-CoV-2 S protein may therefore be expressed as one polypeptide or as more than one polypeptide. In one embodiment the eukaryotic expression cassette encodes at least one polypeptide comprising at least 3 immune-dominant epitopes of SARS-CoV-2 S protein. The immune-dominant epitopes comprised within the at least one or more polypeptide(s) are 3 or more, 5 or more, 10 or more, 20 or more, 30 or more, 50 or more, or even more than 50 immune-dominant epitopes. In the context of the Salmonella typhi Ty21a strain as used herein, the eukaryotic expression cassette encoding the at least 3 immune-dominant epitopes of SARS-CoV-2 S protein may encode one polypeptide comprising up to 50 immune-dominant epitopes or even more, such as up to 300. Antigens presented as peptides on MHC class I or II (in humans HLA) are typically from 11 to 30 amino acids long for MHC II (CD4 antigens) and from 8 to 10 amino acids for MHC I (CD8 antigens).

Thus, preferred ranges for immune-dominant epitopes to be contained within the at least one polypeptide are 3 to 300, 5 to 300, 10 to 300, 20 to 300 or 50 to 300 immune-dominant epitopes. Thus, the polypeptide may further comprise immune-dominant epitopes form other structural proteins of SARS-CoV-2, such as of the E protein, the M protein or the N protein, preferably of the N-protein. Preferred ranges for immune-dominant epitopes of SARS-CoV-2 S protein to be expressed by the eukaryotic expression cassette or to be contained within the at least one polypeptide are 3 to 25, 3 to 20 or 5 to 15. Each polypeptide comprising fused immune-dominant epitopes is proteolytically cleaved into the epitopes inside antigen presenting cells and presented via HLA to elicit a T-cell response.

Given the close genetic similarity between the S protein of SARS-CoV-2 with SARS-CoV (76%), SARS-CoV-2 T and B epitopes may be predicted using pre-existing immunological studies of SARS-CoV (Ahmed et al, Viruses, 2020). T and B cell epitopes may also be predicted using bioinformatic approaches with validated algorithms to recognize amino acid motifs that bind to MHC class I and class II proteins of various HLA molecule (Grifoni et al, Cell, 2020). Public resources such as Immune Epitope Database and Analysis Resource (IEDB), NetMHCPan, and NetMHCIIPan can be used to generate putative T and B cell epitopes. Using these approaches, a multi-epitope vaccine may be designed to encompass sections of the S protein that are rich in epitopes. One region of particular interest is the Receptor Binding Motif (RBM) of the S protein which interacts with the angiotensin-converting enzyme 2 (ACE2) receptor on human target cells to facilitate viral entry. Antibodies towards the RBM of SARS-CoV are neutralizing, however the RBM of SRS-CoV and SARS-CoC-2 has only 50% shared identity and the antibodies do not cross-neutralize (Ju et al, BioRxiv, 2020—submitted; Walls et al, Cell, 2020).

According to the invention, the at least 3 immune-dominant epitopes of SARS-CoV-2 S protein may comprise CD8 T cell antigens and/or CD4 T cell antigens. Preferably, the at least 3 immune-dominant epitopes of SARS-CoV-2 S comprise CD8 T cell antigens and CD4 T cells antigens.

An immune-dominant epitopes is typically a peptide having 8 to 30 amino acids, preferably 8 to 20, more preferably 8 to 12 amino acids.

For vaccine comprising immune-dominant epitopes of SARS-CoV-2 S protein it is beneficial if the vaccine targets multiple immune-dominant epitopes of the S protein, preferably additionally even of further structural proteins, such as the N protein, as this reduces the risk of immune-evasion due to mutations in the S proteins.

Alternatively in certain embodiments the DNA vaccine comprises a Salmonella typhi Ty21a strain comprising a DNA molecule comprising a eukaryotic expression cassette encoding from N-terminal to C-terminal at least three immune-dominant epitopes of SARS-CoV-2 S protein and optionally an enhancer sequence, a 2A peptide or an IRES sequence, an optional ubiquitin sequence and another SARS-CoV-2 protein or a portion thereof, preferably a SARS-CoV-2 N protein or a portion thereof. The portion of the SARS-CoV-2 N protein may be at least three immune-dominant epitopes of SARS-CoV-2 N protein.

Advantage of DNA vaccine according to the invention comprising Salmonella typhi Ty21a, as carrier for the at least SARS-CoV-2 S protein or a portion thereof (such as the 3 immune-dominant epitopes of SARS-CoV-2 S protein, the full-length S protein, the S protein ectodomain, the S protein subunit S1 or the S protein RBD) are the established quality control assay, the individual differences of the plasmid only in the insert encoding the antigen, no need for expansion and no requirements with regard to sterility testing due to oral administration. Furthermore, expression plasmids suitable for transformation as well as the Salmonella typhi Ty21a strain as carrier allow a large insert such as the full-length S protein or a high number of immune-dominant epitopes. It further allows to further introduce another SARS-CoV-2 protein or a portion thereof, such as the SARS-CoV-2 N protein or a portion thereof linked via a 2A peptide or an IRES sequence to the SARS-CoV-2 S protein or a portion thereof.

The immune-dominant epitopes of SARS-CoV-2 S protein (or optionally also N protein) may be inserted into the plasmid as a string of beads (expressed as one or more polypeptides), optionally separated by a linker. The linker may be, without being limited thereto, a GS linker, a 2A cleavage site, or an IRES sequence. Due to the fast generation and only limited need for quality control, the time for generating the Salmonella typhi Ty21a strain comprising a DNA molecule comprising at least one eukaryotic expression cassette encoding the SARS-CoV-2 S protein or a portion thereof is short and can for example be achieved within 15 days, preferably within 14 days or less after identification of the antigen, including immune-dominant epitopes or new clinical isolates or mutants. Overnight fermentation is sufficient and no upscaling is required due to high yield of bacteria with a net yield in the range of 10¹¹ colony forming units (CFU) in a 1 L culture. This allows for a short manufacturing time, as well as the low manufacturing costs. Furthermore, the drug product was shown to be stable for at least three years. Thus, this DNA vaccine is suitable for fast development and production of an effective SARS-CoV-2 prophylactic and/or therapeutic vaccine for use in a large number of subjects in need thereof. Moreover, it is easy to store and does not need medical trained personal for administration.

DNA sequences encoding at least a SARS-CoV-2 S protein or a portions thereof may be separated from DNA sequences encoding the another SARS-CoV-2 protein or a portions thereof with the use of a linker which may be, without being limited thereto, a GS linker, a 2A cleavage site, or an IRES sequence.

Methods for detecting immune-dominant epitopes in a protein and reliably predicting or determining those peptides with high-affinity binding of autologous human leukocyte antigen (HLA) molecules are known in the art. Peptides are then selected that are predicted to likely bind to autologous HLA-A or HLA-B proteins of the patient or which is predominant in the population. This may be confirmed, e.g., by ex vivo interferon γ enzyme-linked immunospot (ELISPOT).

In certain embodiments, the DNA molecule or the DNA molecule comprising the at least one eukaryotic expression cassette comprises an antibiotic resistance gene, such as the kanamycin antibiotic resistance gene, an ori, such as the pMB1 ori or the pUC, and a strong promoter, such as a CMV promoter. In particular embodiments, the DNA molecule or the DNA molecule comprising the at least one eukaryotic expression cassette is a plasmid, such as a plasmid based on or derived from the commercially available pVAX1™ expression plasmid (Invitrogen, San Diego, Calif.).

This expression vector may be modified by replacing the high copy pUC origin of replication by the low copy pMB1 origin of replication of pBR322. The low copy modification was made in order to reduce the metabolic burden and to render the construct more stable. The generated expression vector backbone was designated pVAX10.

The expression vector may also be designed to contain enhancers such as ubiquitin or complement to promote presentation of antigens in MHC class I or II molecules. Plasmid vectors encoding MHC class I antigens and ubiquitin delivered by Salmonella typhimurium to murine have demonstrated enhanced antigen-specific T cell responses and tumour control in a B16 tumour challenge model (Xiang et al, PNAS, 2000). Antibody responses to B cell epitopes encoded by DNA vectors have been shown to be enhanced by inclusion of three copies of complement protein C3d (SEQ ID NO: 4), which binds to the CR2 (CD21) receptor found on B cells and follicular dendritic cells to enhance antigen-specific B cell activation (Moveseyan, J Neuroimmunol, 2008; Yang, Virus Res, 2010; Hou, Virology J, 2019).

Several methods have been used to facilitate translation of multiple genes using a single plasmid vector, including inserting a Internal Ribosome Entry Site (IRES) (Ma et al, Hum Vaccin Immunother, 2013) or 2A peptides between peptide gene sequences (Liu et al, Scientific Reports, 2017).

In particular embodiments, the expression plasmid comprises the DNA molecule of SEQ ID NO: 2 (vector backbone pVAX10), which correlates to the sequence of expression vector pVAX10 without the portion of the multiple cloning site which is located between the restriction sites NheI and XhoI. In one embodiment the expression plasmid comprises a nucleic acid sequence of SEQ ID NO: 2 and a sequence encoding the amino acid sequence of SEQ ID NO:1 or a portion thereof or an amino acid sequence that has at least 95% sequence identity with SEQ ID NO: 1 or a portion thereof.

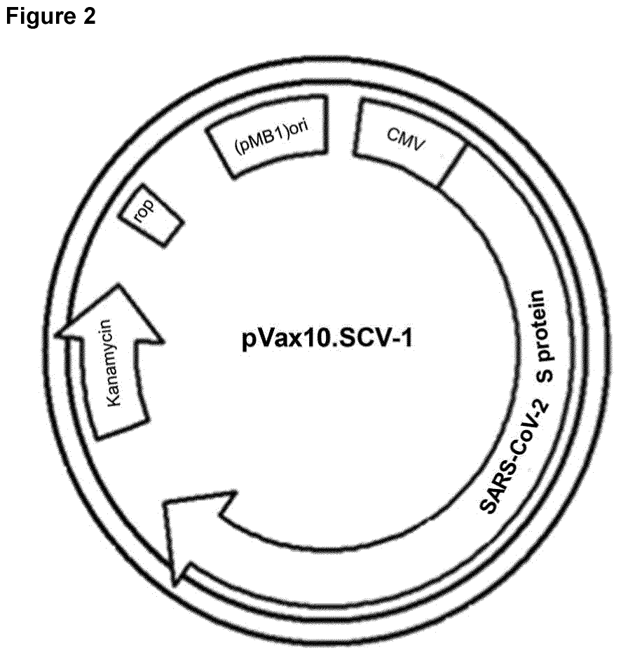

Inserting SARS-CoV-2 S protein encoding ORF with a nucleic acid sequence encoding SEQ ID NO: 1 into this expression vector backbone via NheI/XhoI yielded the expression plasmid. The expression plasmid pVAX10.SCV-1 is schematically depicted in FIG. 2.

The DNA vaccine according to the invention may be in the form of a pharmaceutical composition. Thus, in certain embodiments the DNA vaccine comprising a Salmonella typhi Ty21a strain comprising a DNA molecule comprising a eukaryotic expression cassette encoding at least a COVID-19 coronavirus (SARS-CoV-2) spike (S) protein or a portion thereof further comprise one or more pharmaceutically acceptable excipients. In certain embodiments the DNA vaccine is an oral dosage form. The DNA vaccine of the present invention may be in the form of a solution, a suspension or any other form suitable for the intended oral use. Alternative dosage forms are an enteric coated capsule or a lyophilized powder. Typically, the DNA vaccine according to the present invention is provided as drinking solution, preferably as a suspension, more preferably as an aqueous suspension. This embodiment offers the advantage of improved patient compliance and allows for rapid, feasible and affordable mass vaccination programs, especially in poor geographies.

The invention also provides a pharmaceutical composition comprising the DNA vaccine according to the invention.

In the context of the present invention, the term “excipient” refers to a natural or synthetic substance formulated alongside the active ingredient of a medication. Suitable excipients include solvents, anti-adherents, binders, coatings, disintegrants, flavors, colors, lubricants, glidants, sorbents, preservatives and sweeteners.

In the context of the present invention, the term “pharmaceutically acceptable” refers to molecular entities and other ingredients of pharmaceutical compositions such as a DNA vaccine that are physiologically tolerable and do not typically produce untoward reactions when administered to a mammal (e.g., human). The term “pharmaceutically acceptable” may also mean approved by a regulatory agency of a Federal or a state government or listed in the U.S. Pharmacopeia or other generally recognized pharmacopeia for use in mammals, and, more particularly, in humans.

In certain embodiments, the DNA vaccine or the pharmaceutical composition according to the present invention is in the form of an enteric coated capsule, a lyophilized powder or a suspension. Suitable suspensions comprise means to neutralize gastric acids at least to a certain degree, i.e. to bring the pH of the gastric juice closer to a pH of 7. Thus, in certain embodiment the suspension is a buffered suspension obtained by suspending the attenuated strain of Salmonella according to the present invention in a suitable buffer, preferably in a buffer that neutralizes gastric acids to at least a certain degree, preferably in a buffer containing 2.6 g sodium hydrogen carbonate, 1.7 g L-ascorbic acid, 0.2 g lactose monohydrate and 100 ml of drinking water.

In certain embodiments, the DNA vaccine of the pharmaceutical composition according to the invention further comprises one or more adjuvants.

In the context of the present invention, the term “adjuvant” refers to an agent that modifies the effect of an active ingredient, i.e. the attenuated strain of Salmonella according to the present invention. Adjuvants may boost the immune response to an antigen, thereby allowing to minimize the amount of administered antigen.

In the context of the present invention, the term “vaccine” refers to an agent which is able to induce an immune response in a subject upon administration. A vaccine can preferably prevent, ameliorate or treat a disease. A vaccine in accordance with the present invention comprises the live attenuated strain of Salmonella typhi, S. typhi Ty21a. The vaccine in accordance with the present invention is a DNA vaccine and hence further comprises at least one copy of a DNA molecule comprising a eukaryotic expression cassette, encoding at least a COVID-19 coronavirus (SARS-CoV-2) spike (S) protein or a portion thereof.

The term “DNA vaccine” or “DNA vaccination” as used herein refers to a vaccine for protecting against or treating a disease or infection by delivery of genetically engineered linear DNA or preferably plasmid(s) containing the DNA sequence encoding the antigen(s), such as the SARS-CoV-2 S protein or a portion thereof, against which an immune response is sought to target cells of the patient in need thereof. Thus, the antigen is produced by target cells and induces an immune response. DNA vaccines have potential advantages over conventional vaccines, including the ability to induce a wider range of immune response types, such as a humoral and/or cell-mediated immune response. The plasmid can be delivered to the tissue by several methods, including the use of injection in saline, gene gun, liposomes or via carriers, such as bacterial and viral vectors. The DNA vaccine according to the invention comprises a Salmonella typhi Ty21a strain as carrier for delivery of the DNA molecule comprising a eukaryotic expression cassette encoding at least a COVID-19 coronavirus (SARS-CoV-2) spike (S) protein or a portion thereof. Preferably the DNA molecule delivered by the live attenuated Salmonella typhi Ty21a strain is a plasmid.

The live attenuated Salmonella strain according to the present invention stably carries a DNA molecule encoding at least a COVID-19 coronavirus (SARS-CoV-2) spike (S) protein or a portion thereof. It can be used as a vehicle for the oral delivery of this DNA molecule. Such a delivery vector comprising a DNA molecule encoding a heterologous antigen, such as SARS-CoV-2 S protein or a portion thereof, is referred to as DNA vaccine in the context of the present invention.

Genetic immunization might be advantageous over conventional vaccination. The target DNA can be detected for a considerable period of time thus acting as a depot of the antigen. Sequence motifs in some plasmids, like GpC islands, are immunostimulatory and can function as adjuvants furthered by the immunostimulation due to LPS and other bacterial components.

Live attenuated Salmonella vectors, such as Salmonella typhi Ty21a, produce their own immunomodulatory factors such as lipopolysaccharides (LPS) in situ which may constitute an advantage over other forms of administration such as microencapsulation. Moreover, the mucosal DNA vaccine according to the present invention uses the natural entry site of Coronaviruses, which may prove to be of benefit. The mucosal vaccination has an intra-lymphatic mode of action. After ingestion of the attenuated vaccine according to the present invention, macrophages and other cells in Peyer's patches of the gut are invaded by the modified bacteria. The bacteria are taken up by these phagocytic cells. Due to their attenuating mutations, bacteria of the Salmonella typhi Ty21 strain are not able to persist in these phagocytic cells but die at this time point. The DNA molecules are released from the bacterium and the endosome and are subsequently transferred into the cytosol of the phagocytic immune cells, either via a specific transport system or by endosomal leakage. Finally, the recombinant DNA molecules enter the nucleus, where they are transcribed, leading to massive SARS-CoV-2 S protein expression within the phagocytic cells. The infected cells undergo apoptosis, loaded with the S protein antigen, and are taken up and processed by the gut's immune system. The danger signals of the bacterial infection serve as a strong adjuvant in this process, leading to strong antigen specific CD8+T-cell and antibody responses at the level of both systemic and mucosal compartments. The intra-lymphatic mucosal vaccination route is especially useful for mass vaccinations, and for pathogens that use a mucosal route of entry, such as Coronaviruses.

Salmonella vaccines containing eukaryotic plasmids can generate B cell responses to the antigens encoded by the plasmid. In mice immunized orally with Salmonella typhimurium containing the pCMVb eukaryotic expressed vector encoding antigens listeriolysin or ActA, antigen-specific antibodies could be detected in blood serum by 4 weeks post immunization (Darji et al, Cell, 1997; Darji et al, FEMS Immunol Med Microbiol, 2000).

The vaccine strain Salmonella typhi Ty21a, has an unparalleled safety track record. There is no data available indicating that Salmonella typhi Ty21a is able to enter the bloodstream systemically. The live attenuated Salmonella typhi Ty21a vaccine strain thus allows specific targeting of the immune system in the gut, while being safe and well-tolerated. In contrast, adenovirus-based DNA vaccines might bear an inherent risk of unintended virus replication. In addition, preexisting immunity against adenoviruses was shown to limit vaccine efficacy in humans.

Also provided herein is the DNA vaccine according to the invention for use in the treatment and/or the prevention of coronavirus disease 2019 (COVID-19) or a SARS-CoV-2 infection. Also provided herein is a method for treating and/or preventing coronavirus disease 2019 (COVID-19) or a SARS-CoV-2 infection comprising administering the DNA vaccine according to the invention to a patient in need thereof.

Should adverse events occur that resemble hypersensitivity reactions mediated by histamine, leukotrienes, or cytokines, treatment options for fever, anaphylaxis, blood pressure instability, bronchospasm, and dyspnoea are available. Treatment options in case of unwanted T-cell derived auto-aggression are derived from standard treatment schemes in acute and chronic graft vs. host disease applied after stem cell transplantation. Cyclosporin and glucocorticoids are proposed as treatment options.

In the unlikely case of systemic Salmonella typhi Ty21a type infection, appropriate antibiotic therapy is recommended, for example with fluoroquinolones including ciprofloxacin or ofloxacin. Bacterial infections of the gastrointestinal tract are to be treated with respective agents, such as rifaximin.

In preferred embodiments, the DNA vaccine comprising the Salmonella typhi Ty21a strain comprising a DNA molecule comprising a eukaryotic expression cassette encoding at least a COVID-19 coronavirus (SARS-CoV-2) spike (S) protein or a portion thereof according to the invention is administered orally. Oral administration is simpler, safer and more comfortable than parenteral administration. Although the DNA vaccine of the present invention may also be administered by any other suitable route, the oral route is preferred. Preferably, a therapeutically effective dose is administered to the subject, and this dose may depend on the particular application, particularly whether the DNA vaccine is for therapeutic or prophylactic use, the subject's weight, age, sex and state of health, the manner of administration and the formulation, etc. Administration may be single or multiple, as required.

The DNA vaccine according to the present invention may be provided in the form of a solution, a suspension, lyophilisate, an enteric coated capsule, or any other suitable form. Typically, the attenuated strain of Salmonella according to the present invention is formulated as drinking solution. This embodiment offers the advantage of improved patient compliance. Preferably, the drinking solution comprises means to neutralize gastric acids at least to a certain degree, i.e. to bring the pH of the gastric juice closer to a pH of 7. Preferably, the drinking solution is a buffered suspension comprising the attenuated strain of Salmonella according to the present invention. In a particular embodiment, the buffered suspension is obtained by suspending the attenuated strain of Salmonella according to the present invention in a suitable buffer, preferably containing 2.6 g sodium hydrogen carbonate, 1.7 g L-ascorbic acid, 0.2 g lactose monohydrate and 100 ml of drinking water.

In particular embodiments, the treatment and/or prevention of COVID-19 or a SARS-CoV-2 infection may further comprises administration of a further SARS-CoV-2 vaccine or an anti-SARS-CoV-2 treatment. The treatment and/or prevention of COVID-19 and/or a SARS-CoV-2 infection may further comprises a DNA vaccine comprising a Salmonella typhi Ty21a strain comprising a DNA molecule comprising a eukaryotic expression cassette encoding at least another SARS-CoV 2 protein or a portion thereof, such as a COVID-19 coronavirus (SARS-CoV-2) envelope (E) protein, membrane (M) protein, or nucleocapsid (N) protein or a portion thereof, preferably a SARS-CoV-2 N protein or a portion thereof. The two DNA vaccines may be co-administered or may be administered subsequently, preferably the two DNA vaccines are co-administered.

In certain embodiments, the treatment and/or the prevention of COVID-19 and/or a SARS-CoV-2 infection comprises a prime/boost vaccination against SARS-CoV-2. In the context of the present invention, the term “prime/boost vaccination” refers to an immunization regimen that comprises immunizing a subject with a prime vaccination and subsequently with at least one boost vaccination. In preferred embodiments, the prime vaccine and the boost vaccine are the same; i.e. the prime/boost vaccination represents a homologous prime/boost vaccination. Particularly, the DNA vaccine according to the present invention is administered as prime vaccine and as boost vaccine. In other embodiments, the prime vaccine and the boost vaccine represent different types of vaccines against the same pathogen; i.e. the prime/boost vaccination represents a heterologous prime/boost vaccination. In certain embodiments, the DNA vaccine according to the present invention may be administered as prime vaccine and a further SARS-CoV-2 vaccine is administered as boost vaccine. In particular other embodiments, the further betacoronavirus vaccine is administered as prime vaccine and the attenuated Salmonella strain according to the present invention is administered as boost vaccine. The prime/boost vaccination may elicit superior immune responses than vaccination with a single prime vaccination alone. Improved initial T-cell responses, antibody responses and/or longevity of the immune responses may be achieved by prime/boost vaccination.

In certain embodiments, administration of the prime and the boost DNA vaccine according to the invention occurs within eight consecutive weeks, more particularly within three to six consecutive weeks. Prime vaccine and boost vaccine may be administered via the same route or via different routes. Preferably the prime and the boost DNA vaccine according to the invention are administered via the same route, more preferably the prime and the boost DNA vaccine are administered orally. Also, the DNA vaccine according to the invention may be administered one or several times at the same or different dosages. It is within the ability of the person skilled in the art to optimize prime/boost vaccination regimes, including optimization of the timing and dose of vaccine administration.

In particular embodiments, a single dose of the DNA vaccine comprises the Salmonella typhi Ty21a strain according to the invention at about 10⁵ to about 10¹¹ or at about 1×10⁶ to about 1×10¹⁰, more preferably at about 1×10⁶ to about 1×10⁹, at about 1×10⁶ to about 1×10⁸, or at about 1×10⁶ to about 1×10⁷ colony forming units (CFU). In one embodiment, a single dose of DNA vaccine comprises the Salmonella typhi Ty21a strain at about 1×10⁶ to about 1×10⁹ colony forming units (CFU). Administration of low doses of this live attenuated bacterial DNA vaccine minimizes the risk of excretion and thus of transmission to third parties. It has previously been shown no excretion is detectable below 1×10⁹ CFU.

In this context, the term “about” or “approximately” means within a factor of 3, alternatively within a factor of 2, including within a factor of 1.5 of a given value or range.

In certain embodiments, the treatment and/or the prevention of COVID-19 or a SARS-CoV-2 infection comprises multiple administrations of the DNA vaccine according to the present invention. The single dose of the DNA vaccine administrations may be the same or different, preferably the single dose is the same and comprises the Salmonella typhi Ty21a strain at about 1×10⁶ to about 1×10⁹ colony forming units (CFU). In particular, the treatment and/or the prevention of COVID-19 or a SARS-CoV-2 infection comprises 1, 2, 3, 4, 5 or 6 administrations of the DNA vaccine according to the present invention. Preferably, the treatment and/or prevention of COVID-19 or a SARS-CoV-2 infection comprises that the DNA vaccine is to be administered two to four time in one week for priming (as prime vaccination), optionally followed by one or more single dose boosting. In certain embodiments the DNA vaccine is to be administered 2 to 4 times within the first week (as prime vaccination), followed by one or more single dose boosting each at least 2 weeks later (as boost vaccination), i.e., prime vaccination in the first week and a single dose boost vaccination in week three or later, optionally followed by one (or more) further single dose boost vaccination at least 2 weeks later. In an alternative embodiment the DNA vaccine is to be administered 2 to 4 times within the first week (as prime vaccination), followed by one or more single dose boosting each at least 4 weeks later (as boost vaccination), i.e., prime vaccination in the first week and a single dose boost vaccination in week five or later, optionally followed by one (or more) further single dose boost vaccination at least 4 weeks later.

### EXAMPLES

### Example 1: Preparation of Recombinant Plasmid pVAX10.SCV-1

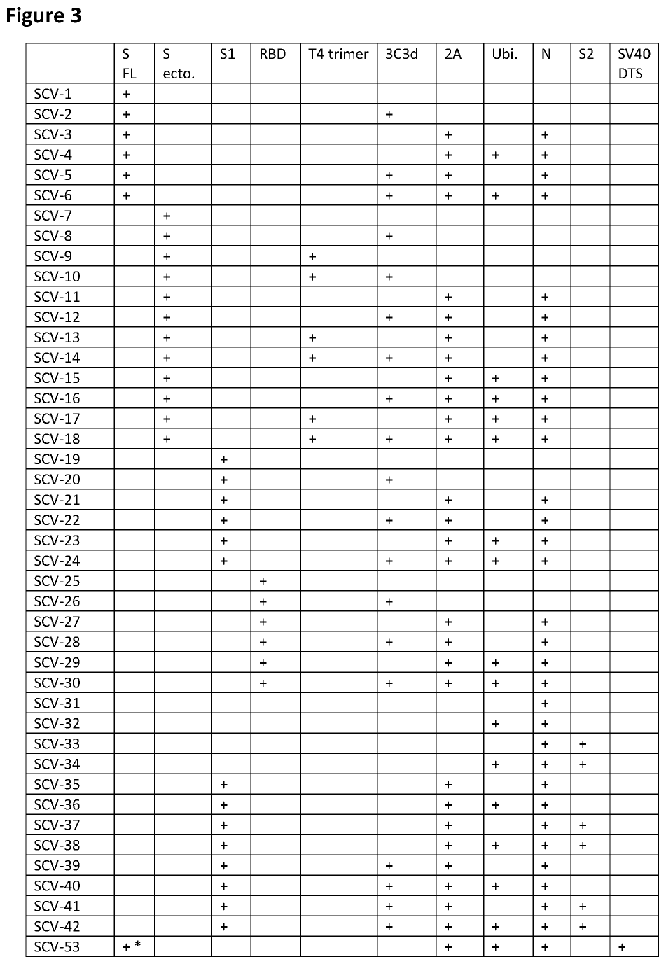

DNA encoding SARS-CoV-2 S protein (1273 aa, SEQ ID NO: 1) is cloned into the pVAX10 backbone derived of pVAX10.VR2-1 (WO 2013/091898). S protein DNA fragments are generated by double-strand gene synthesis, where oligonucleotides are linked together using a thermostable ligase. The obtained ligation products are amplified by PCR. Upon amplification, the in vitro synthesized S protein DNA fragment is cloned into the pVAX10 backbone via NheI/XhoI (the VEGFR-2 coding region of recombinant plasmid pVAX10.VR2-1 is replaced by the S protein coding region). For quality control, the entire plasmid is sequenced and aligned to the respective reference sequence after transformation into E. coli to show that it proves to be free of errors. The resulting plasmid is designated pVAX10.SCV-1 (FIG. 2). Other suitable constructs are shown in FIG. 3.

### Example 2: Transformation of Attenuated Salmonella Strains with the Recombinant Plasmid pVAX10.SCV-1

S. typhi Ty 21a is transformed with plasmid pVAX10.SCV-1. The transformation is performed by electroporation.

### Preparation of Competent Salmonella Cells:

Glycerol cultures of S. typhi Ty21a were inoculated on LB plates (animal component free [ACF] soy peptone). The plates were incubated at 37° C. overnight. One colony was used for overnight-liquid-preculture. 3 ml LB medium (ACF soy peptone) inoculated with one colony was incubated at 37° C. and 180 rpm overnight. To prepare competent cells, 2×300 ml of LB medium (ACF soy peptone) were inoculated with 3 ml of the overnight culture and incubated at 37° C. and 180 rpm up to an OD₆₀₀ of about 0.5. The cultures were then put on ice for 10 minutes. Subsequently, the bacteria were centrifuged for 10 minutes at 3000×g at 4° C. and each pellet was resuspended in 500 mL of ice cold H₂O_{dest}. After a new centrifugation step, the bacterial pellets were washed twice in 10% ice cold glycerol. Both pellets were put together in 2 ml of 10% glycerol and finally frozen in aliquots of 50 μL on dry ice. The used glycerol did not contain any animal ingredients (Sigma Aldrich, G5150).

### Transformation of Competent Salmonella Cells:

For each transformation reaction, a 50 μl aliquot of competent S. typhi Ty21a cells are thawed on ice for 10 minutes. After adding 3-5 μL of plasmid DNA pVAX10.SCV-1 the mixtures is incubated on ice for five minutes. Subsequently, the mixtures are transferred to pre-cooled cuvettes (1 mm thickness). The electric pulse is carried out at 12.5 kV/cm. Immediately afterwards, 1 ml of LB medium (ACF soy peptone) is added to the cells, the cells are transferred into a 2 ml Eppendorf tube and shaken for 1 hour at 37° C. After a short centrifugation step on a bench centrifuge (16600 rcf, 20 s), the bacterial pellet is resuspended in 200 μl of LB (ACF soy peptone) antibiotic-free medium. The mixtures is applied with a Drigalski spatula on LB plates (ACF soy peptone) containing kanamycin (concentration=25 μg/ml or 50 μg/ml). The plates are incubated at 37° C. overnight.

### Plasmid Preparation of Recombinant Salmonella Clones:

Three clones of the recombinant Salmonella typhi Ty21a strain are incubated overnight in 3 ml of LB medium (ACF soy peptone) containing kanamycin (50 μg/ml) at 37° C. The bacterial culture is then pelleted by centrifugation (16600 rcf, 30 s). Plasmid isolation is performed using the NucleoSpin Plasmid Kit from Macherey-Nagel. The plasmid DNA is eluted from the silica gel columns with 50 μl water. 5 μl of the eluate is used in agarose gel electrophoresis for control.

For long-term storage, 1 ml glycerol cultures of the positive clones are produced. For this purpose, 172 μl glycerol (no animal ingredients) are added to 828 μl medium of a logarithmically growing 3 ml culture in a 1 low ml screw microtube. The samples are stored at −70° C. until further use.

Complete Sequencing of Recombinant Plasmid DNA Isolated from Salmonella:

3 ml of liquid LB-Kan medium (ACF soy peptone) are inoculated with one colony of recombinant Salmonella (S. typhi Ty21a harboring pVAX10.SCV-1) and incubated overnight at 37° C. and 180 rpm. The overnight culture is pelleted by centrifugation at 1300 rpm for 30 s on a bench centrifuge (Biofuge pico, Heraeus). The plasmid isolation is performed with the NucleoSpin Plasmid Kit from Macherey-Nagel. After alkaline lysis and precipitation of high molecular weight genomic DNA and cellular components, the plasmid DNA is loaded onto columns with a silica membrane. After a washing step, the plasmids are eluted from the column with 50 μl of sterile water and sequenced. The sequences are then compared with the respective reference sequence by clone specific alignments, i.e. the plasmid sequences of each Salmonella clone is one by one aligned with the reference sequence to check whether all sequences are in line with the respective reference sequences. The recombinant Salmonella strain is designated VXM-SCV-1 (S. typhi Ty21a harboring plasmid pVAX10.SCV-1).

### Example 3: Lame-Scale Production of VXM-SCV-1

Bacterial fermentation is carried out as described in WO 2013/091898. Down-stream processing consists of diafiltration, dilution and filling. One 1001 fermentation run yields approximately 5 liters of 1-10×10¹⁰ CFU/ml of vaccine. The vaccine is further diluted into suitable aliquots and stored at −70° C. The aliquots can be shipped on dry ice. On site, the aliquots are diluted into an application buffer to yield the ready to use vaccine (a 100 ml drinking solution, prepared in bulk).

### Example 4: Preclinical Study Design—Assessing Immune Responses Elicited by VXM-SCV-1 in Healthy Mice

Immune responses against SARS-CoV-2 in healthy C57Bl/6, BALBc or CD1 mice are evaluated by antibody ELISA. Mice are vaccinated with Salmonella typhimurium containing plasmid pVAX10.SCV-1 (10⁸-10⁹ CFU/dose). Salmonella typhimurium containing plasmid pVAX10.SCV-1 are prepared as described above for Salmonella typhi Ty21a. As negative control, a vector control group (10⁸-10¹⁰ CFU/dose Salmonella typhimurium containing no expression plasmid) is included in the study setup to discriminate the desired immunologic effect from any unspecific background stimulation caused by Salmonella empty vector. Immune monitoring is carried out at one or more post-vaccination time points.

### 1. Animal Maintenance

Healthy female mice, 6 weeks old at reception, are observed for 7 days in a specific-pathogen-free (SPF) animal care unit before starting the procedure. Animals are maintained in rooms under controlled conditions of temperature (23±2° C.), humidity (45±10%), photoperiod (12 h light/12 h dark) and air exchange. Animals are maintained in SPF conditions. Room temperature and humidity are continuously monitored. The air handling system is programmed for 14 air changes/hour, with no recirculation. Fresh outside air is passed through a series of filters, before being diffused evenly into each room. A positive pressure (20±4 Pa) is maintained in the experimentation room to prevent contamination or the spread of pathogens within a rodent colony. Animals are housed in polycarbonate cages (Techniplast, Limonest, France) that are equipped to provide food and water. The standard area cages used are 800 cm² with a maximum of 10 mice per cage (from the same group). Bedding for animals is sterile corn cob bedding (ref: LAB COB 12, SERLAB, Cergy-Pontoise, France), replaced twice a week. Animal food is purchased from DIETEX (Saint-Gratien, France). Irradiated RM1 is used as sterile controlled granules. Food is provided ad libitum from water bottles equipped with rubber stoppers and sipper tubes. Water bottles are sterilized by sterile filtration and replaced twice a week. At D0, mice are distributed according to their individual body weight into 2 groups using Vivo manager® software (Biosystemes, Couternon, France). The mean body weight of the two groups (which are then divided into groups 1 to 5 and of groups 6 to 10, respectively) is not statistically different (analysis of variance).

### 2. Detecting Antibody Responses in Mice

BALBc and CD1 mice are divided into six groups of eight. Mice in groups 1-3 receive administration of the vector control, mice in groups 4-6 receive administration of Salmonella typhimurium containing plasmid pVAX10.SCV-1. Both Salmonella typhimurium strains are thawed and administered within 30 min, the working solutions are discarded after use. The treatment dose is 10⁸ CFU in 100 μl per administration. The Salmonella strains are administered by oral gavage (per os, PO) via a cannula with a volume of 0.1 ml. Regardless of animal groups, each animal receives pre-dose application buffer to neutralize acid in the stomach prior dosing (100 μl/animal/application). This buffer is produced by dissolution of 2.6 g sodium hydrogen carbonate, 1.7 g L-ascorbic acid, 0.2 g lactose monohydrate in 100 ml of drinking water and is applied within 30 min prior application of the Salmonella typhimurium strains. The treatment schedule is as follows:

The mice in groups 1 (n=8) and 4 (n=8) receive 3 PO administrations of respective Salmonella typhimurium at 10⁸ CFU every two weeks (Q2WK×3)

The mice in groups 2 (n=8) and 5 (n=8) receive daily PO administrations respective Salmonella typhimurium at 10⁸ in CFU every two days for four consecutive times (Q2D×4).

The mice in groups 3 (n=8) and 6 (n=8) receive daily PO administrations respective Salmonella typhimurium at 10⁸ in CFU every two days for four consecutive times (Q2D×4) and then two boosters every two weeks (Q2WK×2).

The viability and behavior of the animals is recorded every day, body weights are measured twice a week. Serum is collects on weeks 3, 4, 8, 12, 16, 20, 24 and 28 of study and stored at −20° C. until analysis. An autopsy (macroscopic examination of heart, lungs, liver, spleen, kidneys and gastrointestinal tract) is performed on all terminated animals at the end of the study.

Briefly, a 96-well EIA plate is coated overnight with 1 microgram per milliliter of N or S protein epitopes or recombinant whole N or S proteins in sodium carbonate buffer (pH 9.5) at 4° C. Next day, plate is washed with 100 millimolar tris-buffered saline/Tween (TBST) and blocked for 1 hour at 37° C. with 3% gelatin. Plate is thoroughly washed with TBST then serum is added to the top row of each plate and 1:1 dilutions prepared down each column with TBST. On each plate, a negative control column is included with no serum. The plate is incubated overnight at 4° C. To develop, plates are washed with TBST and incubated with 1:1000 dilution of Protein G conjugated to alkaline phosphatase (Calbiochem, USA) for 1 hour at 37° C. The OD405 is measured with an ELISA plate reader. Antibody end-point titre is determined as the reciprocal of the dilution required to give 1 standard deviation OD405 above the average OD405 of the negative control.

### 3. Detecting T Cell Responses in C57BL6 or BALBc Mice

BALBc and C57BL6 mice are divided into six groups of twelve. Mice in groups 1-3 receive administration of the vector control, mice in groups 4-6 receive administration of Salmonella typhimurium containing plasmid pVAX10.SCV-1. Both Salmonella typhimurium strains are thawed and administered within 30 min, the working solutions are discarded after use. The treatment dose is 10⁸ CFU in 100 μl per administration. The Salmonella strains are administered by oral gavage (per os, PO) via a cannula with a volume of 0.1 ml. Regardless of animal groups, each animal receives pre-dose application buffer to neutralize acid in the stomach prior dosing (100 μl/animal/application). This buffer is produced by dissolution of 2.6 g sodium hydrogen carbonate, 1.7 g L-ascorbic acid, 0.2 g lactose monohydrate in 100 ml of drinking water and is applied within 30 min prior application of the Salmonella typhimurium strains. The treatment schedule is as follows:

The mice in groups 1 (n=12) and 4 (n=12) receive 3 PO administrations of respective Salmonella typhimurium at 10⁸ CFU every two weeks (Q2WK×3)

The mice in groups 2 (n=12) and 5 (n=12) receive daily PO administrations respective Salmonella typhimurium at 10⁸ in CFU every two days for four consecutive times (Q2D×4).

The mice in groups 3 (n=12) and 6 (n=12) receive daily PO administrations respective Salmonella typhimurium at 10⁸ in CFU every two days for four consecutive times (Q2D×4) and then two boosters every two weeks (Q2WK×2).

The viability and behavior of the animals is recorded every day, body weights are measured twice a week. One third of the mice in each group (n=4) were euthanized at 14 days, one third (n=4) were euthanized at 28 days, and the remaining one third of the mice (n=4) were euthanized at day 56. At the time of termination spleens and blood samples were collected. Blood was processed for serum, which was stored at −20° C. until analysis. Spleens were processed into a single cell suspension. The immunogenicity of the vaccines was evaluated in the splenocyte preparations by IFN-gamma ELISPOT. Briefly, splenocytes were loaded into wells of an ELISPOT plate pre-coated with anti-IFN-gamma (500,000 cells in 0.1 ml). Peptide epitopes from the N or S protein were added to wells in duplicate at 10 micrograms per milliliter. Plates were incubated at 37° C. for 18 hours. Next day, plates were developed using AEC kits (Sigma, USA) and individual IFN-gamma secreting cells enumerated using an Immunospot plate reader (Cellular Technologies Ltd, USA). Antibodies were detected in the serum samples by ELISA. Briefly, a 96-well EIA plate was coated overnight with 1 microgram per milliliter of N or S protein epitopes or recombinant whole N or S protein in sodium carbonate buffer (pH 9.5) at 4° C. Next day, plate was washed with 100 millimolar tris-buffered saline/Tween (TBST) and blocked for 1 hour at 37° C. with 3% gelatin. Plate was thoroughly washed with TBST then serum was added to the top row of each plate and 1:1 dilutions prepared down each column with TBST. On each plate, a negative control column was included with no serum. The plate was incubated overnight at 4° C. To develop, plates were washed with TBST and incubated with 1:1000 dilution of Protein G conjugated to alkaline phosphatase (Calbiochem, USA) for 1 hour at 37° C. The OD405 was measured with an ELISA plate reader. Antibody end-point titre was determined as the reciprocal of the dilution required to give 1 standard deviation OD405 above the average OD405 of the negative control.

### 4. Antigen Expression Analysis

Antigen expression analysis is performed by transfecting plasmid pVAX10.SCV-1 into murine 3T3 and human 293T cells. At 24 hours and 48 hours after infection, the cells are harvested and lysed. The obtained whole cell lysates are analyzed by SDS poly-acrylamide gel electrophoresis (SDS-PAGE), followed by Western blotting onto a PVDF membrane. RNA expression will also be confirmed by RT/PCR.

### Example 5: Preclinical Study—Assessing Immune Responses Elicited by VXM-SCV-3 in Healthy Mice

The pVAX10-SCV-3 plasmid (insert SCV-3; SEQ ID NO: 11) encodes SARS-CoV-2 spike protein (SEQ ID NO: 1) with the furin domain removed (amino acid residues 680-683) and the SARS-CoV-2 N protein (SEQ ID NO: 8) (Accession no YP_009724397). The antigens are separated by a 2A self-cleaving peptide sequence (SEQ ID NO: 7) derived from capsid protein precursor of Thosea asigna virus (see FIG. 3).

Salmonella typhimurium SL7207 vaccines containing pVAX10-SCV-3 were prepared by electroporation. Competent bacteria were incubated on ice with 100-500 ng of plasmid DNA then electroporated in GenePulsar II at 2.5 kiloVolts. Bacteria were incubated in SOC media for 1 hour at 37 degrees Celsius on a shaker plate, then 100 uL were plated on TSB agar plates with 50 ug/mL kanamycin overnight at 37 degrees Celsius. Individual colonies were expanded and frozen in 25% glycerol at −80 degrees Celsius.

Pathogen free, female BALBc mice, 4-6 weeks of age were purchased from Charles River Laboratories (St Constant, PQ, Canada) and were housed according to institutional guidelines with food and water ad libitum.

A group of 10 mice was treated with the SL-SCV-3 vaccine. For each treatment mice were pre-treated with 100 microliter dose of administration buffer (310 millimolar sodium bicarbonate, 100 millimolar L-ascorbic acid, 5 millimolar lactose monohydrate) by oral gavage, then received 100 microliter dose of vaccine in administration buffer at 1.5-2×10e9 CFU per milliliter. Mice were treated on days 0, 2, 5, 7, 21, and 35. Mice were bled before the study (pre-immune) then on weeks 2, 4, 6 and 8.

Vaccine efficacy was assessed by enzyme-linked immunosorbent assay (ELISA), a method that allows the detection of antigen-specific antibody levels in the serum of immunized animals. Briefly, a 96-well EIA plate was coated with antigen SARS-CoV-2 spike protein (ACROBiosystems) overnight at 4 degrees Celsius, blocked with 2% bovine serum albumin for 1 hour at 37 degrees Celsius, then incubated overnight at 4 degrees Celsius with serial dilutions of sera, typically starting at a dilution of 1/200. A secondary reagent (Goat anti-mouse IgG (H+L) Peroxidase, Jackson ImmunoResearch) was then added to each well at a 1/5000 dilution and incubated for one hour at 37 degrees Celsius. Plates were washed thoroughly and 3,3′,5,5′-Tetramethylbenzidine substrate (Life Technologies) was added to the wells for 5-10 minutes, the reaction was stopped by adding 0.16N H2SO4. The absorbance of each well at 450 nanometers was measured using a microtiter plate reader (Cytation5, Biotek). Endpoint titers were calculated as described in Frey A. et al (Journal of Immunological Methods, 1998, 221:35-41). Calculated titers represented the highest dilution at which a statistically significant increase in absorbance is observed in serum samples from immunized mice versus serum samples from naïve, non-immunized control mice.

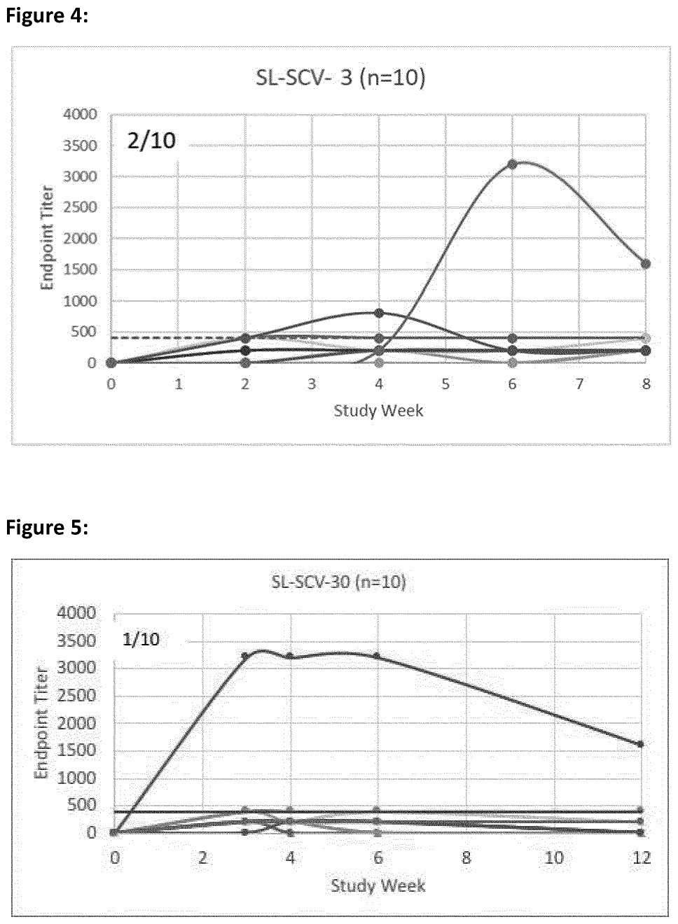

Of the 10 mice vaccinated with SL-SCV-3, 2 mice generated antibody responses greater than assay background of 1/400. One mouse achieved peak antibody titer of 1/800 by week 4 and one mouse achieved and maintained peak antibody titer of 1/3200 by week 6 (see FIG. 4). This demonstrates that a salmonella-based SARS-CoV2 vaccine construct targeting the spike protein is able to generate an antigen-specific immune response against the spike protein, i.e. to generate a humoral immune response.

### Example 6: Preclinical Study—Assessing Immune Responses Elicited by VXM-SCV-30 in Healthy Mice

The pVAX10-SCV-30 plasmid (insert SCV-30; SEQ ID NO: 12) encodes SARS-CoV-2 RBD domain of the spike protein (amino acid 319-541 of SEQ ID NO: 1), followed by three repeats of murine C3d (3C3d; SEQ ID NO: 17; KFLNTAKDRNRWEEPDQQLYNVEATSYA) then 2A self-cleaving peptide sequence (SEQ ID NO: 7) derived from capsid protein precursor of Thosea asigna virus, followed by ubiquitin (SEQ ID NO: 9) fused to the SARS-CoV-2 N protein (SEQ ID NO: 8)(Accession no. YP_009724397) (see FIG. 3).

Salmonella typhimurium SL7207 vaccines were prepared with pVAX10-SCV-30 as described in example 5.

Pathogen free, female BALBc mice, 4-6 weeks of age were purchased from Charles River Laboratories (St Constant, PQ, Canada) and were housed according to institutional guidelines with food and water ad libitum.

A group of 10 mice was treated with the SL-SCV-30 vaccine. For each treatment mice were pre-treated with 100 microliter dose of administration buffer (310 millimolar sodium bicarbonate, 100 millimolar L-ascorbic acid, 5 millimolar lactose monohydrate) by oral gavage, then received 100 microliter dose of vaccine in administration buffer at 1.5-2×10e9 CFU per milliliter. Mice were treated on days 0, 2, 5, 7, 21, and 35. Mice were bled before the study (pre-immune) then on weeks 3, 4, 6, and 12.

Serum was analysed for antibodies towards SARS-CoV-2 spike protein as described in Example 5.

Of the 10 mice vaccinated with SL-SCV-30, one mouse generated antibody responses greater than assay background of 1/400 and reaching 1/3200 by week 3 (see FIG. 5). This demonstrates that a salmonella-based SARS-CoV2 vaccine construct targeting the RBD domain of the spike protein is able to generate an antigen-specific immune response against the spike protein.

### Example 7: Preclinical Study—Assessing Immune Responses Elicited by VXM-SCV-42 in Healthy Mice

The pVAX10-SCV-42 plasmid (insert SCV-42; SEQ ID NO: 13) encodes SARS-CoV-2 S1 domain of the spike protein (amino acid 1-681 of SEQ ID NO: 1), followed by three repeats of murine C3d (SEQ ID NO: 17; 3C3d, SEQ ID NO: 18) then 2A self-cleaving peptide sequence (SEQ ID NO: 7) derived from capsid protein precursor of Thosea asigna virus, followed by ubiquitin (SEQ ID NO: 9) fused to the SARS-CoV-2 N protein (SEQ ID NO: 8) another 2A self-cleaving peptide sequence and SARS-CoV-2 S2 domain of the spike protein (Ser686-Thr1273 of SEQ ID NO: 1) (see FIG. 3).

Salmonella typhimurium SL7207 vaccines were prepared with pVAX10-SCV-42 as described in example 5.

Pathogen free, female BALBc mice, 4-6 weeks of age were purchased from Charles River Laboratories (St Constant, PQ, Canada) and were housed according to institutional guidelines with food and water ad libitum.

A group of 10 mice was treated with the SL-SCV-42 vaccine. For each treatment mice were pre-treated with 100 microliter dose of administration buffer (310 millimolar sodium bicarbonate, 100 millimolar L-ascorbic acid, 5 millimolar lactose monohydrate) by oral gavage, then received 100 microliter dose of vaccine in administration buffer at 1.5-2×10e9 CFU per milliliter. Mice were treated on days 0, 2, 5, 7, 21, and 35. Mice were bled before the study (pre-immune) then on weeks 2, 4, 6, and 8.

Serum was analysed for antibodies towards SARS-CoV-2 spike protein as described in Example 5.

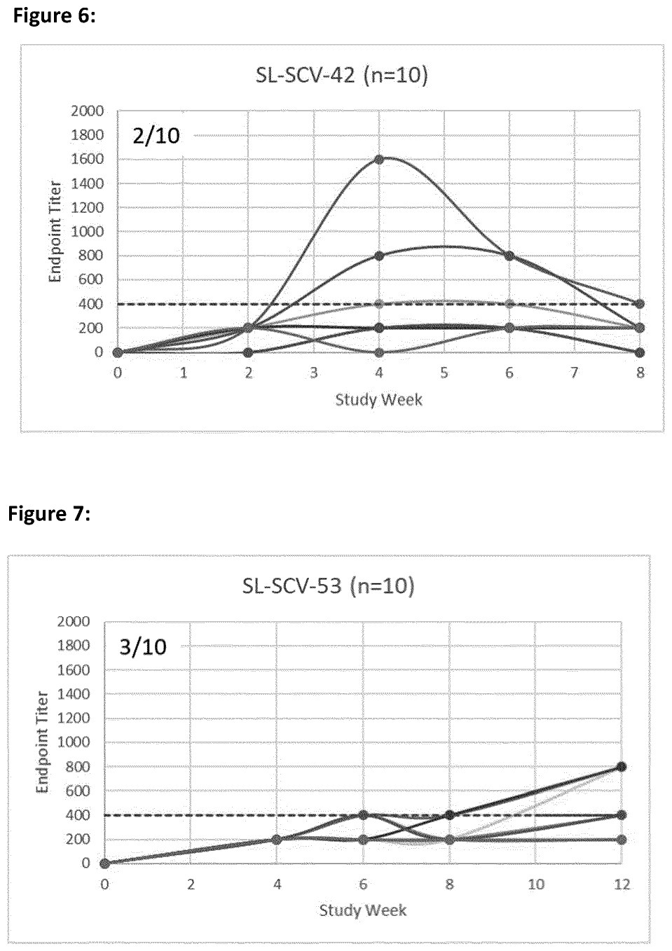

Of the 10 mice vaccinated with SL-SCV-42, 2 mice generated antibody responses greater than assay background of 1/400 and reaching 1/1600 (see FIG. 6). This demonstrates that a salmonella-based SARS-CoV2 vaccine construct targeting the S1 and/or the S2 subunit of the spike protein is able to generate an antigen-specific immune response against the spike protein.

### Example 8: Preclinical Study—Assessing Immune Responses Elicited by VXM-SCV-53 in Healthy Mice

The pVAX10-SCV-53 plasmid (insert SCV-53; SEQ ID NO: 14; entire plasmid sequence SEQ ID NO: 19) encodes SARS-CoV-2 spike protein (SEQ ID NO: 1) with the furin domain removed (amino acid residues 680-683 deleted) and where the signal domain (Met1-Ser12 of SEQ ID NO: 1) has been replaced with that of invariant chain (Met1-Arg29 of SEQ ID NO: 15), followed by 2A self-cleaving peptide sequence (SEQ ID NO: 7) derived from capsid protein precursor of Thosea asigna virus, followed by ubiquitin (SEQ ID NO: 9) fused to the SARS-CoV-2 N protein (SEQ ID NO: 8). The plasmid also contains the 72 nucleotide SV40 DNA nuclear targeting sequence (DTS) (SEQ ID NO: 16) within a larger SV40 ori enhancer sequence (SEQ ID NO: 20) upstream of the kanamycin resistance gene (see FIG. 3).

Salmonella typhimurium SL7207 vaccines were prepared with pVAX10-SCV-53 as described in example 5.

Pathogen free, female BALBc mice, 4-6 weeks of age were purchased from Charles River Laboratories (St Constant, PQ, Canada) and were housed according to institutional guidelines with food and water ad libitum.

A group of 10 mice was treated with the SL-SCV-53 vaccine. For each treatment mice were pre-treated with 100 microliter dose of administration buffer (310 millimolar sodium bicarbonate, 100 millimolar L-ascorbic acid, 5 millimolar lactose monohydrate) by oral gavage, then received 100 microliter dose of vaccine in administration buffer at 1.5-2×10e9 CFU per milliliter. Mice were treated on days 0, 2, 5, 7, 21, and 35. Mice were bled before the study (pre-immune) then on weeks 2, 4, 6, and 8.

Serum was analysed for antibodies towards SARS-CoV-2 spike protein as described in Example 5.

Of the 10 mice vaccinated with SL-SCV-53, 3 mice generated antibody responses greater than assay background of 1/400 and reaching 1/800 (see FIG. 7). This demonstrates that a salmonella-based SARS-CoV2 vaccine construct targeting a signal domain modified spike protein is able to generate an antigen-specific immune response against the spike protein.

### Example 9: VXM-SCV-X Phase I Clinical Trial; Study Design

The aim of this phase I trial is to examine the safety, tolerability, and immunological responses to VXM-SCV-X. The randomized, placebo-controlled, double blind dose-escalation study includes 45 subjects. The subjects receive four doses of VXM-SCV-X or placebo on days 1, 3, 5, and 7. Doses from 10⁶ CFU up to 10⁹ CFU of VXM-SCV-X are evaluated in the study. An independent data safety monitoring board (DSMB) is involved in the dose-escalation decisions. In addition to safety as primary endpoint, the VXM-SCV-1-specific immune reactions are evaluated.

The objectives are to examine the safety and tolerability, and immunological responses to the investigational anti-SARS-CoV-2 virus vaccine VXM-SCV-X, as well as to identify the maximum tolerated dose (MTD) of VXM-SCV-1. The MTD is defined as the highest dose level at which less than two of up to six patients under VXM-SCV-X treatment experience a dose-limiting toxicity (DLT).

Primary endpoints for safety and tolerability are adverse events and serious adverse events according to the CTCAE criteria.

Secondary endpoints, which assess the efficacy of the experimental vaccine to elicit a specific immune response to SARS-CoV-2 S protein, include the number of immune positive patients.

VXM-SCV-X is manufactured according to Good Manufacturing Practice (GMP) and is given in a buffered solution. The placebo control consisted of isotonic sodium chloride solution.

The starting dose consists of a solution containing 10⁶ colony forming units (CFU) of VXM-SCV-X. This VXM-SCV-X dose was chosen for safety reasons. For comparison, one dose of Typhoral®, the licensed vaccine against typhoid fever, contains 2×10⁹ to 6×10⁹ CFU of Salmonella typhi Ty21a, equivalent to approximately thousand times the VXM-SCV-1 starting dose. The dose is escalated in logarithmic steps, which appears to be justified for a live bacterial vaccine.

Complying with guidelines for first-in-human trials, the patients of one dose group are treated in cohorts. The first administration of VXM-SCV-X in any dose group is given to one patient. The second cohort of each dose group consists of two patients receiving VXM-SCV-X. This staggered administration with one front-runner, i.e. only one patient receiving VXM-SCV-X first, serves to mitigate the risks.

A third cohort of patients (three receiving VXM-SCV-X are included in all dose groups.

The environmental risk inherent to an oral vaccine is the potential of excretion to the environment and subsequent vaccination of people outside the target population. All study patients are confined in the study site for the period during which vaccinations take place plus three additional days. All feces of study patients are collected and incinerated. Body fluids and feces samples are investigated for VXM-SCV-X shedding.

Hygienic precautions are applied to protect study personnel from accidental uptake. Study personnel are trained specifically for this aspect of the study.

In addition, specific T-cell activation and antibody formation are measured in this patient setting. A placebo control is included, in order to gain further knowledge on specific safety issues related to the active vaccine vs. the background treatment. In addition, the pooled placebo patients serve as a sound comparator for assessing specific immune activation.

### Example 10: VXM-SCV-1 Specific T-Cell and B Cell Responses

Responses to VXM19 are assessed by monitoring the frequencies of SARS-CoV-2 virus S protein specific T-cells in peripheral blood of VXM-SCV-X and placebo treated patients, detected by IFNγ ELISpot, at different time points prior during and post vaccination.

Firstly, T-cells and peptide pulsed DC are added to wells coated with anti-INFγ antibodies. After a period of incubation, cells are removed with secreted INFγ left binding with the coat antibodies. Then detection antibody is added to detect the bound INFγ, and after a signal amplification, the final yield can be viewed as “color spots” representing single activated and specific T-cells.

B cell responses are measured by ELISA. Briefly, a 96-well EIA plate is coated overnight with 1 microgram per milliliter of N or S protein epitopes or recombinant whole N or S proteins in sodium carbonate buffer (pH 9.5) at 4° C. Next day, plate is washed with 100 millimolar tris-buffered saline/Tween (TBST) and blocked for 1 hour at 37° C. with 3% gelatin. Plate is thoroughly washed with TBST then serum is added to the top row of each plate and 1:1 dilutions prepared down each column with TBST. On each plate, a negative control column is included with no serum. The plate is incubated overnight at 4° C. To develop, plates are washed with TBST and incubated with 1:1000 dilution of Protein G conjugated to alkaline phosphatase (Calbiochem, USA) for 1 hour at 37° C. The OD405 is measured with an ELISA plate reader. Antibody end-point titre is determined as the reciprocal of the dilution required to give 1 standard deviation OD405 above the average OD405 of the negative control.

### Example 11: Anti-Carrier Immunity

In order to assess immune responses to the bacterial vehicle, anti-Salmonella typhi IgG and IgM immunoglobulins are detected by ELISA using two commercial assay kits (Salmonella typhi IgG ELISA, Cat. No. ST0936G and Salmonella typhi IgM ELISA, Cat. No. ST084M; Calbiotech. Inc., 10461 Austin Dr, Spring Valley, Calif. 91978, USA). These assays are qualitative assays. The assays are used as described in the package inserts respectively App. I/I) and as modified as part of the study plan according to the foregoing validation study 580.132.2785.

Both assays employ the enzyme-linked immunosorbent assay technique. Calibrator, negative control, positive control and samples are analyzed as duplicates. Diluted patient serum (dilution 1:101) is added to wells coated with purified antigen. IgG or IgM specific antibody, if present, bind to the antigen. All unbound materials are washed away and the enzyme conjugate is added to bind to the antibody-antigen complex, if present. Excess enzyme conjugate is washed off and substrate is added. The plate is incubated to allow for hydrolysis of the substrate by the enzyme. The intensity of the color generated is proportional to the amount of IgG or IgM specific antibody in the sample. The intensity of the color is measured using a spectrophotometric microtiter plate reader at 450 nm. The cut off is calculated as follows:

Calibrator OD×Calibrator Factor(CF).

The antibody index of each determination is determined by dividing the OD value of each sample by cut-off value.

Antibody Index Interpretation:

| | |
|---|---|
| <0.9 | No detectable antibody to Salmonella typhi |
| | IgG or IgM by ELISA |
| 0.9-1.1 | Borderline positive |
| >1.1 | Detectable antibody to Salmonella typhi |
| | IgG or IgM by ELISA |

### Example 12: Vaccination Schedule

A single dose of VXM19, i.e. from 10⁶ to 10⁸ CFU is administered orally as 100 ml drinking solution. Vaccination with a single dose each occurs on days 1, 3, 5 and optionally 7. Peak immune response are expected to occur around 10 days after the last vaccination. Boosting may be considered after 2 to 4 weeks or even after 3 to 6 months. Schedule recommendations are derived from vaccine strain Ty21a.

1. A DNA vaccine comprising a Salmonella typhi Ty21a strain comprising a DNA molecule comprising a eukaryotic expression cassette encoding at least a COVID-19 coronavirus (SARS-CoV-2) spike (S) protein or a portion thereof.

2. The DNA vaccine according to claim 1, wherein the COVID-19 coronavirus (SARS-CoV-2) spike (S) protein or a portion thereof comprises
(a) a SARS-CoV-2 full-length S protein;
(b) a SARS-CoV-2 S protein ectodomain;
(c) a SARS-CoV-2 S protein subunit S1;
(d) a SARS-CoV-2 S protein receptor binding domain (RBD); or
(e) at least 3 immune-dominant epitopes of SARS-CoV-2 S protein.

3. The DNA vaccine according to claim 2, wherein the COVID-19 coronavirus (SARS-CoV-2) spike (S) protein is a SARS-CoV-2 full-length S protein, optionally wherein the SARS-CoV-2 full-length S protein comprises an amino acid sequence of SEQ ID NO: 1 or an amino acid sequence having at least 95% sequence identity with SEQ ID NO: 1.

4. The DNA vaccine according to claim 2, wherein the COVID-19 coronavirus (SARS-CoV-2) spike (S) protein or a portion thereof comprises the SARS-CoV-2 S protein ectodomain, optionally wherein the SARS-CoV-2 S protein ectodomain comprises an amino acid sequence of amino acid residues 1-1208 of SEQ ID NO: 1 or an amino acid sequence having at least 95% sequence identity with amino acid residues 1-1208 of SEQ ID NO: 1.

5. The DNA vaccine according to claim 2, wherein the COVID-19 coronavirus (SARS-CoV-2) spike (S) protein or a portion thereof comprises the SARS-CoV-2 S protein subunit S1, optionally wherein the SARS-CoV-2 protein subunit S1 comprises an amino acid sequence of amino acid residues 1-681 of SEQ ID NO: 1 or an amino acid sequence having at least 95% sequence identity with amino acid residues 1-681 of SEQ ID NO: 1.

6. The DNA vaccine according to claim 2, wherein the COVID-19 coronavirus (SARS-CoV-2) spike (S) protein or a portion thereof comprises the SARS-CoV-2 S protein receptor binding domain (RBD), optionally wherein the SARS-CoV-2 protein RBD comprises an amino acid sequence of amino acid residues 319-541 of SEQ ID NO: 1 or an amino acid sequence having at least 95% sequence identity with amino acid residues 319-541 of SEQ ID NO: 1.

7. The DNA vaccine according to claim 2, wherein the SARS-CoV-2 S protein or a portion thereof is a prefusion-stabilized form of the SARS-CoV-2 full-length S protein or the SARS-CoV-2 S protein ectodomain comprising two stabilizing mutations to proline corresponding to amino acid position K986 and V987 in the amino acid sequence of SEQ ID NO: 1; preferably wherein the SARS-CoV-2 S protein or a portion thereof comprises
(a) an amino acid sequence of SEQ ID NO: 1 or an amino acid sequence having at least 95% sequence identity with SEQ ID NO: 1, comprising two stabilizing mutations K986P and V987P; or
(b) an amino acid sequence of amino acid residues 1-1208 of SEQ ID NO: 1 or an amino acid sequence having at least 95% sequence identity with amino acid residues 1-1208 of SEQ ID NO: 1, comprising two stabilizing mutations K986P and V987P.

8. The DNA vaccine according to claim 1, wherein the eukaryotic expression cassette further encodes another SARS-CoV-2 protein or a portion thereof.

9. The DNA vaccine according to claim 8, wherein the other SARS-CoV-2 protein is a SARS-CoV-2 N protein.

10. The DNA vaccine according to claim 1, further comprising one or more pharmaceutically acceptable excipients.

11. The DNA vaccine according to claim 1, wherein the vaccine is an oral dosage form.

12. The DNA vaccine according to claim 11, wherein the oral dosage form is an enteric coated capsule, a lyophilized powder or a suspension.

13. The DNA vaccine according to claim 1 further comprising one or more adjuvants.

14. A method of treating and/or preventing coronavirus disease 2019 (COVID-19) or a SARS-CoV-2 infection comprising administering the DNA vaccine according to claim 1.

15. The method according to claim 14, wherein the DNA vaccine is administered orally.

16. The method according to claim 14, wherein
(a) a single dose of DNA vaccine comprises the Salmonella typhi Ty21a strain at about 10⁶ to about 10⁹ colony forming units (CFU), and/or
(b) the DNA vaccine is to be administered 2 to 4 times in one week for priming, optionally followed by one or more single dose boosting.

17. The method according to claim 16, wherein the DNA vaccine is to be administered 2 to 4 times within the first week, followed by one or more single dose boosting each at least 2 weeks later.
